(19) United States
(12) Patent Application Publication
(10) Pub. No.: US 2002/0077754 A1
(43) Pub. Date: Jun. 20, 2002

(54) PHARMACOPHORE FINGERPRINTING IN PRIMARY LIBRARY DESIGN

(76) Inventors: MALCOLM J. MCGREGOR, SUNNYVALE, CA (US); STEVEN M. MUSKAL, SAN JOSE, CA (US)

Correspondence Address: Vicki G. Norton, Esq., BROBECK, PHLEGER & HARRISON LLP, 12390 EL CAMINO REAL, SAN DIEGO, CA, 92130, US

(21) Appl. No.: 09/416,550

(22) Filed: Oct. 12, 1999

Related U.S. Application Data
(60) Provisional application No. 60/145,611, filed on Jul. 26, 1999.
(60) Provisional application No. 60/106,007, filed on Oct. 28, 1998.

(51) Int. Cl. G01N 33/48; C12Q 1/68; G01N 33/53; A01N 37/18

(52) U.S. Cl. 702/19; 514/2; 435/6; 435/7.1

(57) ABSTRACT

Specialized apparatus and methods may be used for identifying, representing, and productively using high activity regions of chemical structure space. At least two representations of chemical structure space provide valuable information. A first representation has many dimensions representing members of a pharmacophore basis set and one or more additional dimensions representing defined chemical activity (e.g., pharmacological activity). A second representation has many fewer dimensions, each of which represents a principle component obtained by transforming the first representation via principal component analysis used on pharmacophore fingerprint/activity data for a collection of compounds. When the collection of compounds has the defined chemical activity, that activity will be reflected as a “high activity” region of chemical space in the second representation.

A “transformation” procedure may convert between the first and second representations. If pharmacophore fingerprints for an “investigation” set of compounds is transformed to the second representation of chemical space, those compounds can be “screened” for high activity. Those compounds residing in the region of high activity may likely have the desired activity. Those compounds residing outside the region probably do not have the desired activity. The compounds falling within high activity region may be selected for a primary library or a more constrained library, depending upon the specificity of the high activity region.

### CROSS REFERENCE TO RELATED APPLICATIONS

This patent application claims priority from U.S. Provisional Application No. 60/145,611 filed Jul. 26, 1999 and from U.S. Provisional Application No. 60/106,007 filed Oct. 28, 1998; both provisional applications name M. J. McGregor and S. M. Muskal as inventors and are incorporated herein by reference; this patent application is also a continuation-in-part of application number ______, filed on Oct. 5, 1999, naming M. J. McGregor and S. M. Muskal as inventors, and titled “PHARMACOPHORE FINGERPRINTING IN QSAR” (docket no. 2069.1) which is incorporated herein by reference for all purposes and in its entirety.

### FIELD OF THE INVENTION

The present invention pertains to methods and apparatus for designing libraries of chemical compounds. More specifically, the present invention relates to the design of primary libraries of chemical compounds. The invention also pertains to defining an active subspace (e.g., a bioactive space) within a general representation of chemical space to assist in designing primary libraries useful in drug discovery, for example.

### BACKGROUND OF THE INVENTION

Recent advances in combinatorial chemistry and high throughput screening have provided experimental access to large collections of compounds (D. K. Agrafiotis et al., Molecular Diversity, 1999, 4, 1-22; U. Eichler et al., Drugs of the Future, 1999, 24, 177-190; A. K. Ghose et al., J. Comb. Chem., 1, 1999, 55-68; E. J. Martin et al., J Comb. Chem., 1999, 1, 32-45; P. R. Menard et al., J. Chem. Inf. Comput. Sci., 1998, 38, 1204-1213; R. A. Lewis et al., J. Chem. Inf. Comput. Sci., 1997, 37, 599-614; M. Hassan et al., Molecular Diversity, 1996, 2, 64-74; M. J. McGregor et al., J. Chem. Inf. Comput. Sci., 1999, 39, 569-574; R. D. Brown, Perspectives in Drug Discovery and Design, 1997, 7/8, 31-49 which are herein incorporated by reference). Consequently, analysis of the calculated properties of large collections of compounds has become increasingly important in drug development. Targeted or focused library design and primary library design are two applications where analysis of the calculated properties of large collections of compounds provides especially relevant information for drug design.

Targeted library design is essentially an extension of the disciplines of computational chemistry and molecular modeling, which may utilize Quantitative Structure Activity Relationships (QSAR) for scaffold design and building block selection. QSAR comprises calculating molecular descriptors, which are used to construct a model that predict biological activity against a single target.

Primary libraries may be used to generate active compounds for one or more targets in the absence of any structural information about either the receptor or the ligand. Primary libraries may be screened against a number of structurally unrelated or diverse targets. In addition, primary libraries could also be used to generate compounds which have optimal absorption, distribution, metabolism, excretion (ADME) and toxicity profiles which are activities unrelated to ligand binding that are important activities of pharmaceutically active molecules.

Finally, an intermediate library may be used to identify compounds active against a family of structurally related compounds. Thus, an intermediate library possesses properties characteristic of both focused libraries and primary libraries.

Identifying a set of descriptors to characterize molecular structure is a crucial step in the analysis of a large set of chemical compounds. A large number of descriptors have been described and can be classified in terms of an approach to molecular structure (M. Hassan et al., Molecular Diversity, 1996, 2, 64-74; M. J. McGregor et al., J. Chem. Inf. Comput. Sci., 1999, 39, 569-574; R. D. Brown, Perspectives in Drug Discovery and Design, 1997, 7/8, 31-49 which were previously incorporated by reference. R. D. Brown et al., J. Chem. Inf. Comput. Sci. 1996, 36, 572-584; R. D. Brown et al., J. Chem. Inf. Comput. Sci. 1996, 37, 1-9; D. E. Patterson et al., J. Med. Chem. 1996, 39, 3049-3059; S. K. Kearsley et al., J. Chem. Inf. Comput. Sci. 1996, 36, 118-127 which are herein incorporated by reference). One dimensional (ID) properties are overall molecular properties such as molecular weight and “clogp.” Two dimensional properties (2D) incorporate molecular functionality and connectivity. A good example of 2D descriptors is the MDL substructure keys, MDL Information Systems Inc., 14600 Catalina St., San Leandro, Calif. 94577 (M. J. McGregor et al., J. Chem. Inf. Comput. Sci., 1997, 37, 443 which is herein incorporated by reference) and the MSI₅₀ descriptors, Molecular Simulations Inc., 9685 Scranton Road, San Diego, Calif. 92121-3752. For example, the well known rule of five that is useful in specifying some requirements for pharmaceutical compounds is derived from one dimensional and two dimensional descriptors (C. A. Lipinski et al., Advanced Drug Delivery Reviews, 1997, 23, 3 which is herein incorporated by reference).

Calculation of three-dimensional descriptors (3D) requires at least an energetically reasonable three dimensional structure. Additionally, contributions from multiple conformations can be considered in the calculation of three-dimensional descriptors. Descriptors can also be chosen on the basis of features important in ligand binding or association with any other important desirable property. Alternatively, when many descriptors are used in an analysis of a large set of chemical compounds, statistical methods such as Principle Component Analysis (PCA) or Partial Least Squares (PLS) can establish a minimal set of important descriptors.

Pharmacophore screening is now a routine method in computer aided drug design (P. W. Sprague et al., Perspectives in Drug Discovery and Design, ESCOM Science Publishers B. V., K. Müller, ed. 1995, 3, 1-20; D. Barnum et al., J. Chem. Inf. Comput. Sci., 1996, 36, 563-571; J. Greene et al., J. Chem. Inf. Comput. Sci., 1994, 34, 1297-1308 which are herein incorporated by reference). Pharmacophore screening is potentially valuable in analyzing large compound collections provided by high throughput screening and combinatorial chemistry. The pharmacophore concept is based on interactions observed in molecular recognition such as hydrogen bonding, ionic and hydrophobic associations. A pharmacophore is defined as a set of functional group types (e.g., aromatic center, negative charge, hydrogen bond donor, etc.) in a specific spatial arrangement (e.g., a triangle) that represents the common interactions between a set of ligands and a biological target. Pharmacophores, by this definition, are 3D descriptors.

Commercially available software systems that perform pharmacophore screening include Catalyst, by Molecular Simulations Inc., 9685 Scranton Road, San Diego, Calif. 92121-3752 (P. W. Sprague, Perspectives in Drug Discovery and Design, ESCOM SciencePublishers B.V., K. Müller, ed., 1995, 3, 1-20; D. Barnum et al., J. Chem. Inf. Comput. Sci., 1996, 36, 563-57; J. Greene et al., J. Chem. Inf. Comput. Sci., 1994, 34, 1297-1308) and the ChemDiverse module of Chem-X by Chemical Design Ltd., Roundway House, Cromwell Park, Chipping Norton, Oxfordshire, OX75SR, U.K (S. D. Pickett et al., J. Chem. Inf. Comput. Sci., 1996, 36, 1214-1223 which is herein incorporated by reference). Unfortunately, the utility of these software systems is limited by required registration of compounds into a closed database system owned by the vendors.

Pharmacophore fingerprinting is an extension of the above approach where enumerating pharmacophoric types with a set of distance ranges provides a basis set of pharmacophores. The basis set of pharmacophores is then applied to a set of compounds to generate pharmacophore fingerprints which are descriptors based on features that are important in ligand-receptor binding. Pharmacophore fingerprinting has been described (A. C. Good et al., J. Comput. Aided Mol. Des., 1995, 9, 373; J. S. Mason et al., Perspective in Drug Discovery and Design. 1997, 718/, 85; S. D. Pickett et al., J. Chem. Inf. Comput. Sci., 1998, 38, 144; S. D. Pickett et al., J. Chem. Inf. Comput. Sci., 1996, 36, 1214-1223; C. M. Murray et al., J. Chem. Inf. Comput. Sci., 1999, 39, 46; J. S. Mason et al., J. Med. Chem., 1999, 39, 46; S. D. Pickett et al., J. Chem. Inf. Comput. Sci., 1998, 38, 144; R. Nilakantan et al., J. Chem. Inf. Comput. Sci., 1993, 33, 79) and applications to structure activity relationships have been reported (X. Chen et al., J. Chem. Inf. Comput. Sci., 1998, 38, 1054). Each of these references is incorporated herein by reference.

A calculated molecular descriptor should possess several desirable features. Ideally a descriptor should provide a quantitative measure of molecular similarity. Association with an experimentally measurable property increases the utility of a molecular descriptor. For example, a calculated logP should approach the measured value as closely as possible. An important property in drug design is ligand binding to a biological target. Ligand binding can be calculated explicitly when the structure of the target is available (e.g., via docking calculations). However, usually ligand binding is typically estimated from more easily calculated properties, which can be regarded as independent variables. Descriptors that contain conformational information should provide superior estimates of biological activity, and 3D descriptors should be better than 2D descriptors. However this has been difficult to demonstrate since sometimes 2D descriptors actually outperform 3D descriptors.

Three dimensional pharmacophore fingerprinting methodology has been applied to relate chemical structure to activity for a single target (M. J. McGregor and S. M. Muskal, “PHARMACOPHORE FINGERPRINTING IN QSAR” U.S. Patent Ser. No. ______ (docket no. 2069.1); M. J. McGregor et al., J. Chem. Inf. Comput. Sci., 1999, 39, 569-574 which were previously incorporated by incorporated by reference). Ligand binding predictions, based on pharmacophore fingerprints were used to provide QSAR for the estrogen receptor that was superior to previously reported studies. Such structure-activity relationships have significant potential in the design of targeted or focused libraries.

The versatile and information-rich nature of pharmacophore fingerprints indicates that this descriptor may also be useful in primary library design. A number of desirable goals can be identified that are related to successful pharmaceutical primary library design. First, a properly designed pharmaceutical primary library should have members active against a number of diverse biological targets. Second, pharmaceutical primary libraries should provide a maximal number of members that bind to a biological target in the absence of any knowledge of either receptor or ligand structure. Third, pharmaceutical primary libraries should provide members that bind to biological targets with high specificity. Finally, pharmaceutical primary libraries should allow for optimization of drug properties such as absorption, distribution, metabolism and excretion that are unrelated to binding to a biological target. Thus, an ideal primary library, in this context, will provide a collection of compounds that have a property distribution similar to compounds that have a measured level of biological activity. Thus a conceptual distinction can be made between chemical space and a subspace thereof, referred to as “bioactive space.” The same distinction can also be made between maximizing molecular diversity and providing optimal coverage of bioactive space.

Regardless of whether a pharmacophore approach is employed, it has become apparent, as new methods of screening with large numbers of compounds becomes increasingly important in modern pharmaceutical research, that developing improved methods that relate a chemical structural descriptor to molecular diversity and properties characteristic of drugs would be highly useful. Thus, what is needed is a computationally efficient method that provides primary libraries that define important properties of bioactive molecules, which can be used to design combinatorial libraries with optimum property distributions.

### SUMMARY OF THE INVENTION

The present invention provides apparatus and methods for identifying, representing and productively using high activity regions of chemical space. Many representations of chemical space have been used and may be envisioned. In a preferred embodiment of this invention, at least two representations provide valuable information. A first representation has many dimensions defined by a pharmacophore basis set and one or more additional dimensions representing defined chemical activity (e.g., pharmacological activity). A second representation may be one of reduced dimensionality, where the coordinates can be derived from the first representation by a suitable mathematical technique such as, for example, the principle components produced by Principle Component Analysis using pharmacophore fingerprint/activity data for a collection of compounds.

A “transformation” procedure may convert between the first and second representations. If pharmacophore fingerprints for an “investigation” set of compounds are transformed to the second representation of chemical space, those compounds can be “screened” for high activity. Those compounds residing in the region of high activity may have the desired activity. Those compounds residing outside the region probably do not have the desired activity. The compounds falling within high activity region may be selected for a primary library or a more constrained library (e.g., a focused library), depending upon the specificity of the high activity region.

One aspect of this invention pertains to identifying one or more regions of a defined activity in a chemical space. First, a “reference” set of compounds having members associated with the defined activity is provided. Second, pharmacophore fingerprints of the reference set are generated. Each fingerprint specifies a three dimensional superposition of pharmacophores from a basis set. Third, the pharmacophore fingerprints of the reference set are associated with the defined activity, which preferably identifies at least one region of the chemical space associated with the defined activity. The process of association may also transform a representation of chemical space to a reduced dimensional space.

In one embodiment, the defined activity is a biological activity such as pharmacological activity. In another embodiment, the defined activity can be properties that are unrelated to binding to a biological target such as absorption, distribution, oral bioavailability, metabolism, and excretion. If the defined activity is pharmacological activity, the reference set should include pharmacologically active compounds. In some embodiments, the reference set is a subset of a database of pharmacologically active compounds. In one specific embodiment, the reference set is the compounds that comprise the MDL Drug Data Report. Alternatively, the reference set may be a subset of the MDL Drug Data Report. Other data sets of biologically active molecules may also be used as a reference set.

In a preferred arrangement, the subset can be prepared from a database of pharmacologically active compounds by selecting compounds within a defined molecular weight range (between about 200 Daltons and about 700 Daltons) that include only carbon, nitrogen, oxygen, hydrogen, sulfur, phosphorus, fluorine, bromine, chlorine and iodine atoms or mixtures thereof. In a more specific embodiment, compounds are eliminated from the subset when the Tanimoto coefficient between a structural representation of the compound and a structural representation of another compound in the database is greater than a defined value (e.g. about 0.8).

Pharmacophore fingerprints employed in this invention may be obtained by the following method: (a) receiving a three-dimensional machine-readable representation of the compound; (b) assigning pharmacophoric types to positions in the three-dimensional representation of the compound, the pharmacophoric types specifying distinct chemical properties; (c) choosing a current conformation of the compound; (d) identifying matches between a current conformation of the compound and a basis set of pharmacophores, each pharmacophore in the basis set having three or more spatially separated pharmacophoric centers with associated pharmacophoric types; and (e) creating the pharmacophore fingerprint from matches of the compound to members of the basis set. Typically, this process will repeat steps (a) through (e) until a pharmacophore fingerprint exists for every member of the reference set. The pharmacophore fingerprint is preferably a bit sequence in which individual bits correspond to unique pharmacophores form the basis set. In a preferred embodiment, the pharmacophoric types assigned to atom positions in the three-dimensional representation of the compound include a hydrogen bond acceptor, a hydrogen bond donor, a center with a negative charge, a center with a positive charge, a hydrophobic center and a default category that does not fall into any other specified pharmacophore type.

Any suitable mathematical technique may be employed to associate the pharmacophore fingerprints of the reference set to the defined activity in a chemical space. A particularly preferred method is Principle Component Analysis, which also reduces the dimensionality of the chemical space. Examples of other suitable techniques include back-propagation neural networks, partial least squares, multiple linear regression and genetic algorithms.

In a preferred arrangement, associating pharmacophore fingerprints with the defined activity transforms a representation of chemical space from a first representation where members of the pharmacophore basis set are the dimensions of a chemical space to a second representation where the principal components are the dimensions of a chemical space. In a more specific embodiment, the compounds of the reference set may be displayed in the second representation of chemical space where the principal components are the dimension axes.

Another aspect of this invention pertains to generating a library of compounds. First, one or more regions of a defined activity are identified in a chemical space (possibly using the above-described process). Second, pharmacophore fingerprints of an investigation set of compounds for the library are provided. Each fingerprint specifies a three dimensional superposition of pharmacophores from a basis set. Third, a subset of the investigation set of compounds having pharmacophore fingerprints falling within the one or more regions of the defined activity is identified. The subset comprises the library of compounds. In a preferred arrangement, a subset of the investigation set of compounds is selected by identifying the members of the investigation set that have substantial overlap with one or more regions of the defined activity in chemical space.

In one embodiment, the library of compounds is a focused library and the defined activity is binding to a particular target. In another embodiment, the library is a primary library and the one or more regions of a defined activity in chemical space are multiple therapeutic activities.

One embodiment of the invention provides a general method of selecting the subset of the members of the investigation set. The method which may be a genetic algorithm may be characterized as including the following sequence: (a) randomly selecting a current subset of the members of the investigation set; (b) calculating an overlap between the current subsets and the reference set within defined regions of the chemical space; (c) selecting, based on calculated overlap, one of the current subset or a previous subset of the members of the investigation set; (d) mutating a selected subset to change its membership; and (e) repeating steps (b) through (d) until the overlap converges. In one example, chemical space is divided into cells by a grid. Overlap is calculated for each cell in the grid and then averaged.

A third aspect of this invention provides a computer program product that pertains to a representation of a chemical space stored on a machine-readable medium. The representation of chemical space identifies chemical compounds by their locations with respect to one or more principal components derived from pharmacophore fingerprints and associated activities for a plurality of compounds from a reference set of compounds. The representation of chemical space identifies one or more regions of a defined activity.

These and other features and advantages of the present invention will be described below in conjunction with the associated figures.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

The invention will be better understood by reference to the following description taken in conjunction with the accompanying drawings in which:

FIG. 1 is a high-level flowchart, which illustrates one approach to generating a library of compounds;

FIG. 2 is a flowchart illustrating one procedure for filtering a database of pharmacologically active compounds to obtain a reference set of compounds;

FIG. 3 is a flowchart that describes a preferred process for generating pharmacophoric fingerprints for a set of compounds;

FIG. 4 illustrates a generalized 3-point pharmacophore;

FIG. 5 illustrates the input representation of a molecular structure used for generating a pharmacophoric fingerprint in accordance with a specific embodiment of this invention;

FIG. 6A is a structural fragment containing a chlorine atom that would be assigned a default pharmacophore type in accordance with an embodiment of this invention.;

FIG. 6B is a chemical structure containing a chlorine atom that would be assigned a hydrophobic pharmacophore type in accordance with an embodiment of this invention;

FIG. 6C is a chemical structure containing a collection of moieties that represent all seven pharmacophore groups in accordance with an embodiment of this invention;

FIG. 7 illustrates a data structure for assigning pharmacophore types to the atoms of acetic acid anion during generation of a pharmacophore fingerprint;

FIG. 8A is a flowchart that depicts a preferred method for generating conformation(s) of a chemical structure during pharmacophore fingerprinting;

FIG. 8B shows a chemical compound with rotatable carbon-carbon sp bonds;

FIG. 8C illustrates the axial and equatorial conformational isomers that may be evaluated for the compound illustrated in FIG. 8B;

FIG. 9 is a flowchart which illustrates a preferred method for calculating overlap or molecular diversity of subsets of the investigation set with a high activity region of chemical space;

FIG. 10 is a block diagram of a generic computer system that may be used with the method and apparatus of the current invention;

FIG. 11 illustrates principle component transformation in matrix form;

FIG. 12 illustrates the 8 combinatorial scaffolds analyzed in Example 5;

FIG. 13A illustrates, in color, the 8 largest target classes in the MDDR9104 set with principle components 1 and 2 as the axes;

FIG. 13B illustrates, in color, the 8 largest target classes in the MDDR9104 set principle components 2 and 3 as the axes;

FIG. 14A illustrates, in color, the number of bits set in the compounds of FIG. 13A with principle components 1 and 2 as the axes;

FIG. 14B illustrates, in color, the presence of formal charges in the compounds of FIG. 13A with principle components 1 and 2 as axes;

FIG. 15 illustrates the results of the AP calculation of Example 4; and

FIG. 16 illustrates molecules from the MDDR9104 that occupy a region of PCA space not covered by the combinatorial libraries in Example 5.

### DETAILED DESCRIPTION OF THE PREFERRED EMBODIMENTS

Reference will now be made in detail to a preferred embodiment of the invention. An example of the preferred embodiment is illustrated in the accompanying drawings. While the invention will be described in conjunction with a preferred embodiment, it will be understood that it is not intended to limit the invention to this preferred embodiment. To the contrary, it is intended to cover alternatives, modifications, and equivalents as may be included within the spirit and scope of the invention as defined by the appended claims.

1. Overview of Library Generation Process

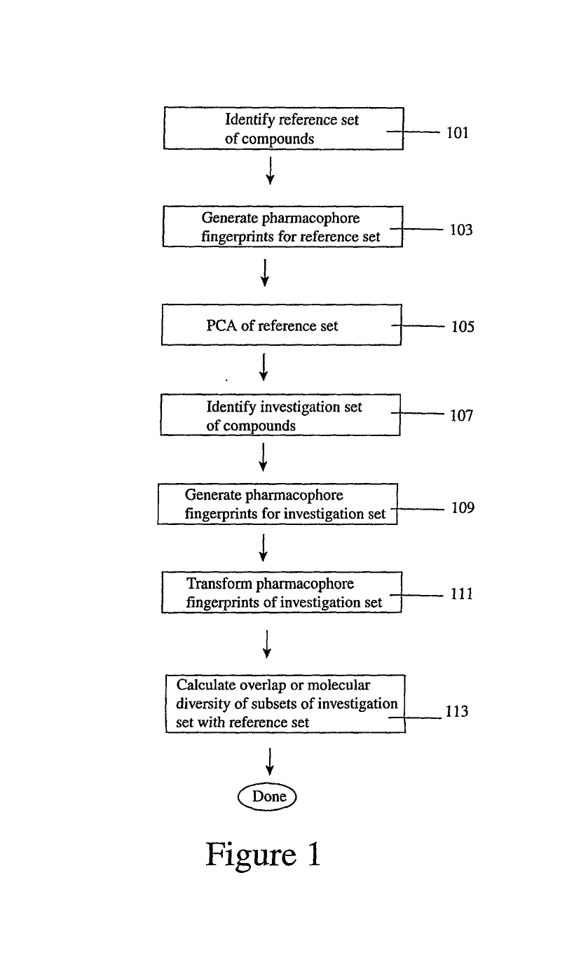

FIG. 1 is a flowchart that illustrates some general steps that may be used to design a library of compounds. A library in the context of this invention will usually be a primary library or, in some situations, a more constrained library (e.g., a focused or targeted library). A focused library is designed for screening against a specific target. A primary library generally subsumes potential ligands for multiple targets. It may be designed for screening against a number of targets which may be unrelated. One important primary library will encompass regions of chemical space inhabited by commercially valuable drugs.

Generally, a primary library may be designed that possesses any useful property or activity exhibited by a collection of chemical compounds. More specifically, for example, a primary library may be comprised of members that have biological or pharmacological activity. In a preferred embodiment, the primary library may have properties characteristic of pharmaceutical compounds that are effective against various human disease states. Particular primary libraries of potential pharmaceutical compounds may be comprised of compounds that have good absorption, distribution, oral bioavailability, metabolism and excretion properties. In alternative embodiments, a primary library may span multiple classes of chemical materials having properties other than pharmacological activity. For example, the primary library may include organic compounds potentially having other biological properties such as herbicidal properties or it may include inorganic materials potentially having properties such as high conductivity, superconductivity, catalytic properties, dielectric properties, luminescence, magnetostrictive properties, ferroelectric properties, and the like. FIG. 1 presents a high-level overview of some important computational processes that may be used in the instant invention.

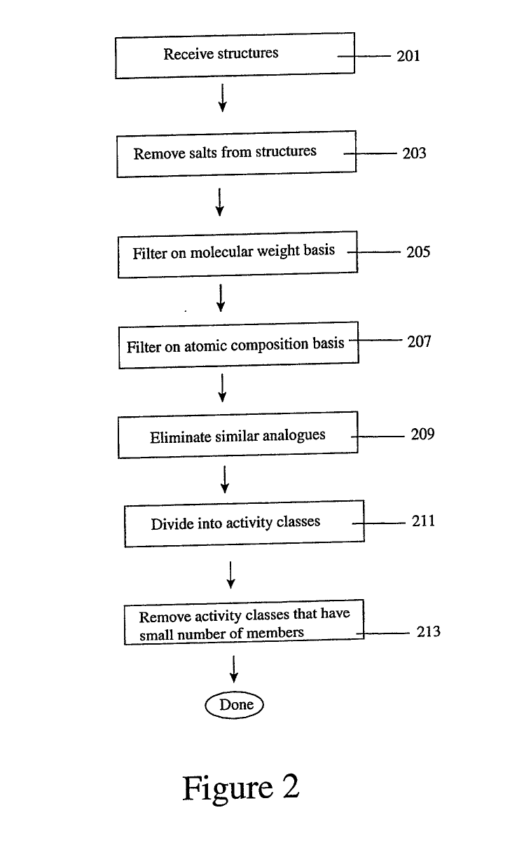

The process of FIG. 1 begins with selecting a reference set that is used as a template for library construction in step 101. Generally, a reference set will be comprised of members that exhibit a defined activity of interest. The reference set may also possess multiple defined activities that are usually related. Ideally, the resulting library will be comprised of members that also exhibit the same defined activity or multiple activities of interest as the reference set. Subsets of compound databases that have especially desirable properties may also be generated and used as the reference set in library design. A detailed process for generating a specific subset from a large collection of compounds will be described in more detail with reference to FIG. 2.

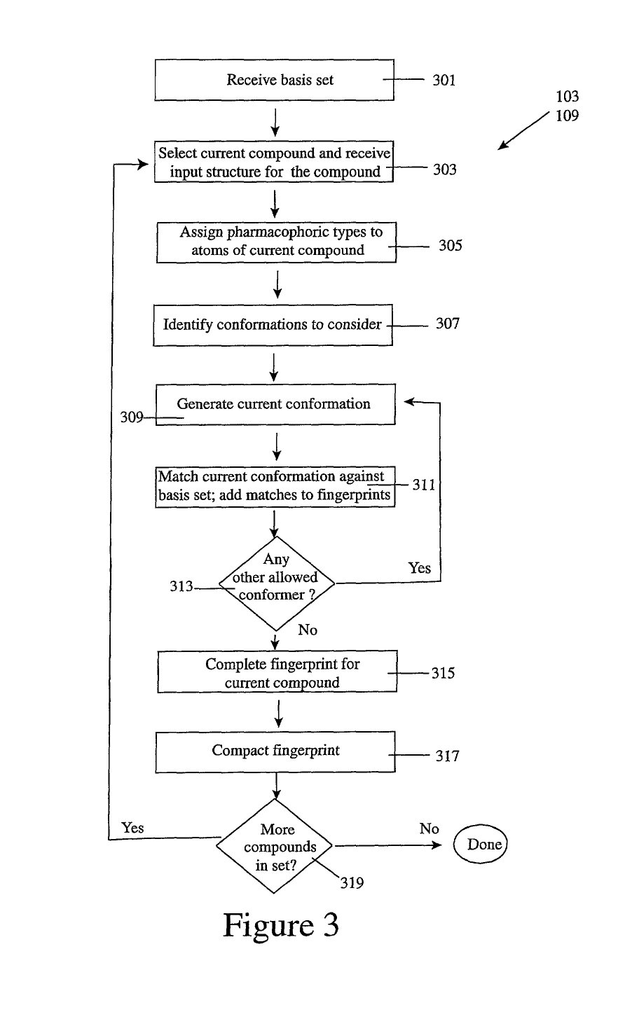

A pharmacophore fingerprint is generated for each member of the reference set in step 103. This process will be described in more detail below with reference to FIG. 3. For now simply recognize that a pharmacophore fingerprint is a convenient method of representing the structure of a compound, over one or more conformations. A fingerprint is generated by matching conformations of a compound of interest against a basis set of pharmacophores.

The pharmacophore fingerprints of the reference set define a region in one representation of chemical space. Each compound of the reference set has a position in the region represented by its pharmacophore fingerprint. Each compound of the reference set may also have a position in a second representation of chemical space created by, for example, Principle Component Analysis of the pharmacophore fingerprints of the reference set compounds and their known activities. In some cases, the second representation may include “principal components” as axes or dimensions. The structures of the reference set compounds will have coordinates in space given by their relative positions along the principal component axes. Importantly, the structural relationship between compounds in the reference set can be defined by their relative position in chemical space. Generally, compounds that are close to one another in chemical space may be structurally similar and, in some cases, may be expected to possess similar activity.

An association between the desired activity and chemical structure can be obtained by defining regions of chemical space where compounds of the desired activity reside. If the first representation of chemical space includes all members of the pharmacophore basis set as independent variables (with a separate dimension or axis for each member), it is typically difficult to visualize or otherwise interpret a region (or regions) of high activity. To facilitate interpretation, the above-mentioned Principle Component Analysis or other methods may be employed to generate the principal components used in the second representation of chemical space.

In a preferred embodiment, the selected mathematical technique reduces the dimensionality of the chemical space. For example, association of the pharmacophore fingerprints with the defined activity or multiple activities in step 105 may produce a reduced set of independent orthogonal descriptors that encompass the information contained in the original data. Thus, association of the pharmacophore fingerprints places the individual members of the reference set in a chemical space where the orthogonal descriptors may represent the dimension axes. Generating this association provides a “transformation” that may be used to map an arbitrary chemical material from a first representation of chemical space (using the basis set of pharmacophores) to a second representation of chemical space (using a reduced dimensionality). Other mathematical techniques that may be used to associate pharmacophore fingerprints to defined activities (without necessarily reducing the dimensionality of chemical space) include back propagation neural networks and genetic algorithms.

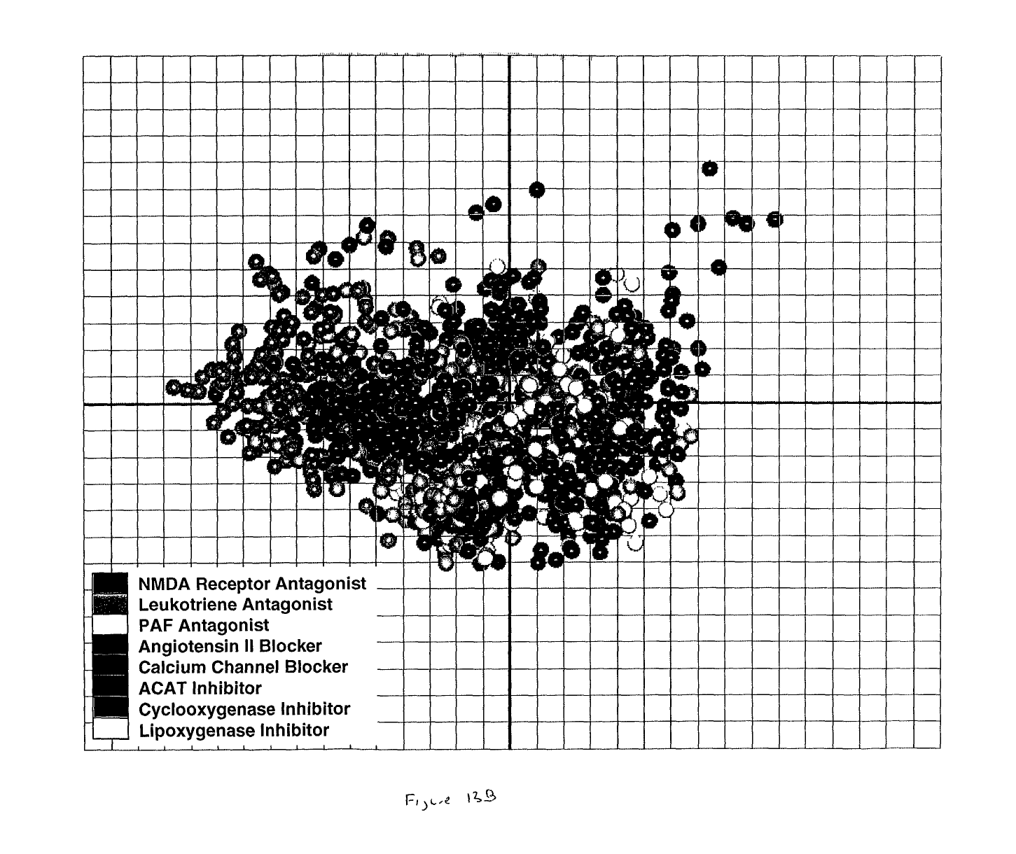

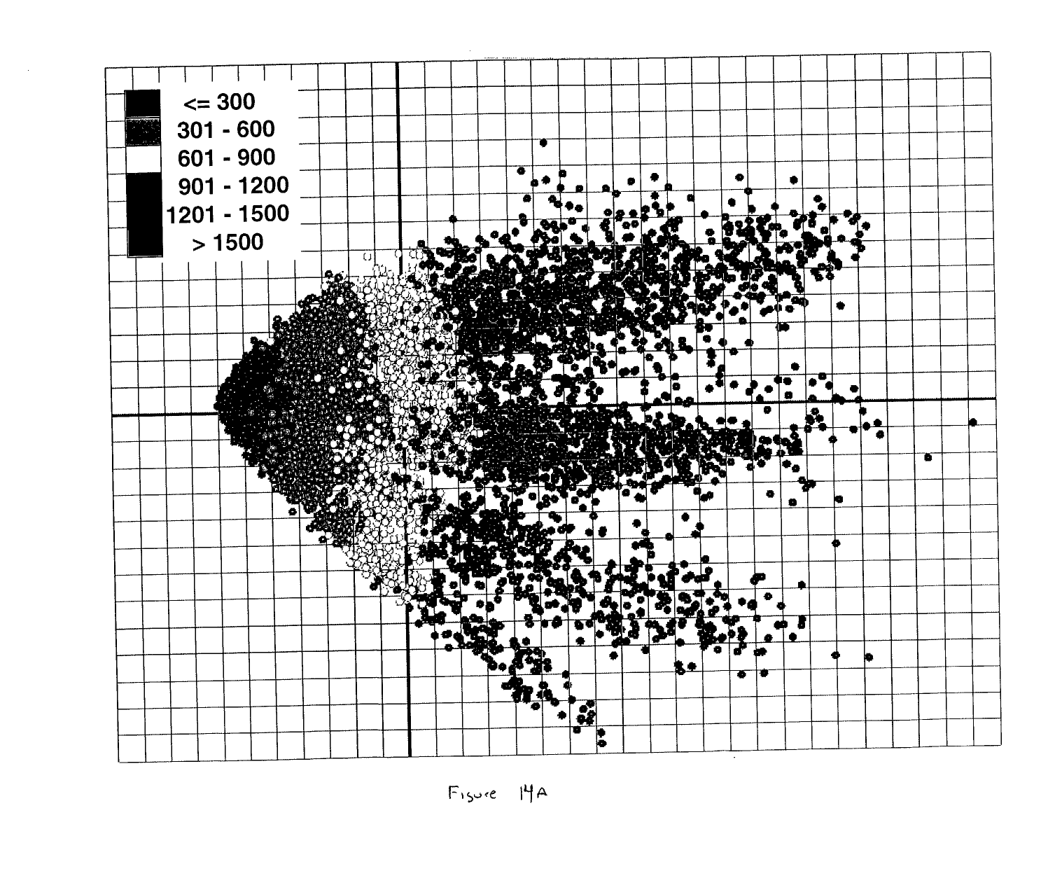

As discussed below, FIG. 13A shows a second representation (specifically a principal component representation) of chemical space having a rather focused region of high activity. The high activity in this case is pharmacological activity. The points in FIG. 13A represent compounds of the reference set having known pharmacological activity. Collectively, they define a region of “high activity.” The horizontal and vertical axes shown in FIG. 13A are principal components obtained by Principle Component Analysis.

Considering again the process depicted in FIG. 1, an investigation set of compounds is identified in step 107. Generally, the investigation set can be any group of compounds. In one specific example, the investigation set is a combinatorial library. Subsets of the investigation set with especially desirable properties may also be identified and used as the investigation set in library design. Ideally, at least a portion of investigation set possess the defined activity or multiple activities exhibited by the reference set members.

Generally, at this stage it is unknown which, if any, of the investigation set members possess the defined activity or multiple activities exhibited by the reference set members. An important goal of the process flow of FIG. 1 is determining which members of the investigation set possess the defined activity or multiple activities exhibited by the reference set members.

In step 109 a pharmacophore fingerprint is provided for each member of the investigation set. In a preferred embodiment, the process of step 109 will not differ from the process of step 103. Pharmacophore fingerprinting, as previously mentioned, will be described in more detail with reference to FIG. 3.

Each compound of the investigation set has a position in chemical space represented by its pharmacophore fingerprint. The structural relationship between compounds in the investigation set may be defined by their relative positions in the chemical space. Similarly, the structural relationship between compounds in the investigation set and the reference set may be defined by their relative positions in the chemical space. As previously mentioned compounds proximate to one another in chemical space may exhibit some structural similarity and therefore may also exhibit some functional similarity.

Part of the process of 105, is transformation of pharmacophore fingerprints. This transformation allows conversion of an arbitrary pharmacophore fingerprint to a coordinate in the second (principal component) representation of chemical space such as that depicted in FIGS. 13A and 13B. The process of FIG. 1 makes use of this at 111 where pharmacophore fingerprints of the investigation set are transformed to coordinates based on principal components. Generally, the transformation, by using Principle Component Analysis for example, in step 111 places the compounds of the investigation set in the second representation of chemical space and allows easy visual comparison with the reference set. At this point, the investigation set of compounds and the reference set of compounds have been projected in the same representation of chemical space (e.g., the representation generated via the mentioned transformation) which may be pictorially represented for rapid comparison.

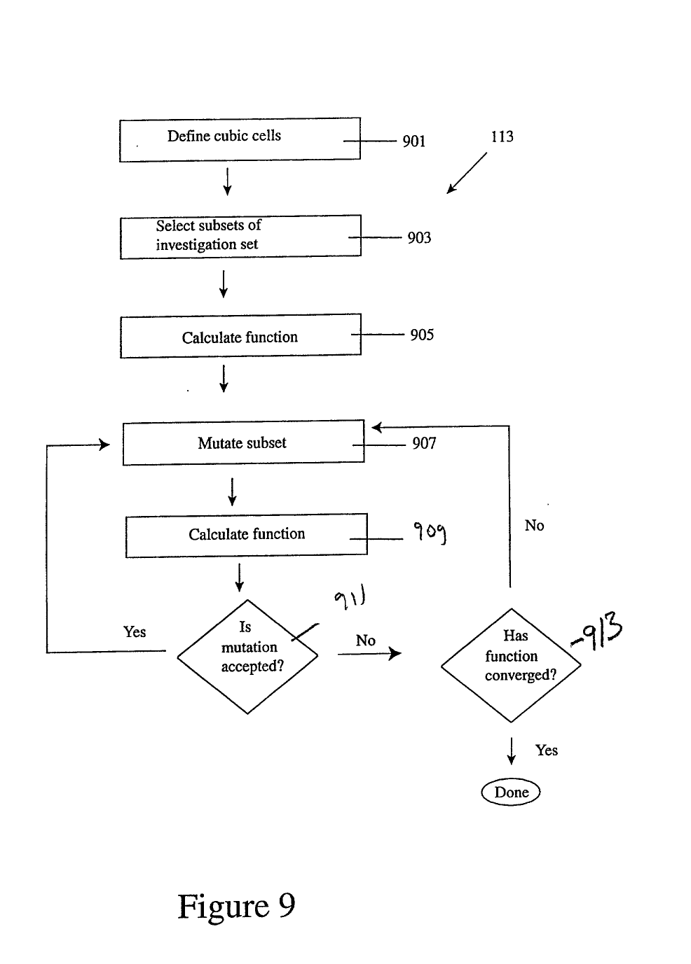

Finally in step 113 the molecular diversity or overlap of subsets of the investigation set with high activity regions of chemical space is calculated. A variety of selection procedures such as cell-based selection, cluster based selection and dissimilarity based selection may be used to select subsets of the investigation set with maximal overlap or molecular diversity with high activity regions of chemical space (see e.g., R. D. Brown et al., Exp. Op. Ther. Patents, 1998, 8(11), 1447 which is herein incorporated by reference). In one embodiment, those investigation compounds lying within the region of high activity associated with reference set are selected. However, when the investigation set is very large, it may be desirable to choose only a subset of such compounds. Further, the region of high activity may not have sharp boundaries and may be somewhat unfocused. In a preferred embodiment, a genetic algorithm is used to select the subset of the investigation set (see e.g., D. E. Goldberg, Genetic Algorithms in Search, Optimization and Machine Learning, Addison Wesley, New York, N.Y. which is herein incorporated by reference). Selection of a subset of the investigation set using a genetic algorithm will be described in more detail with reference to FIG. 9.

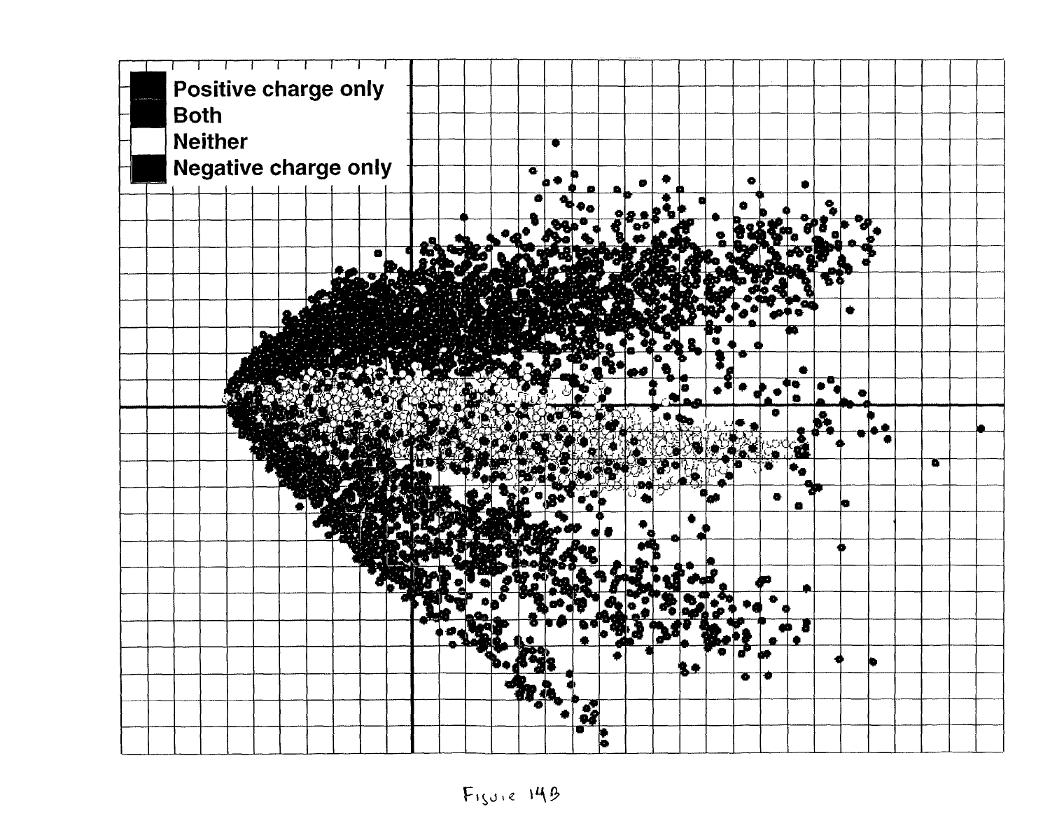

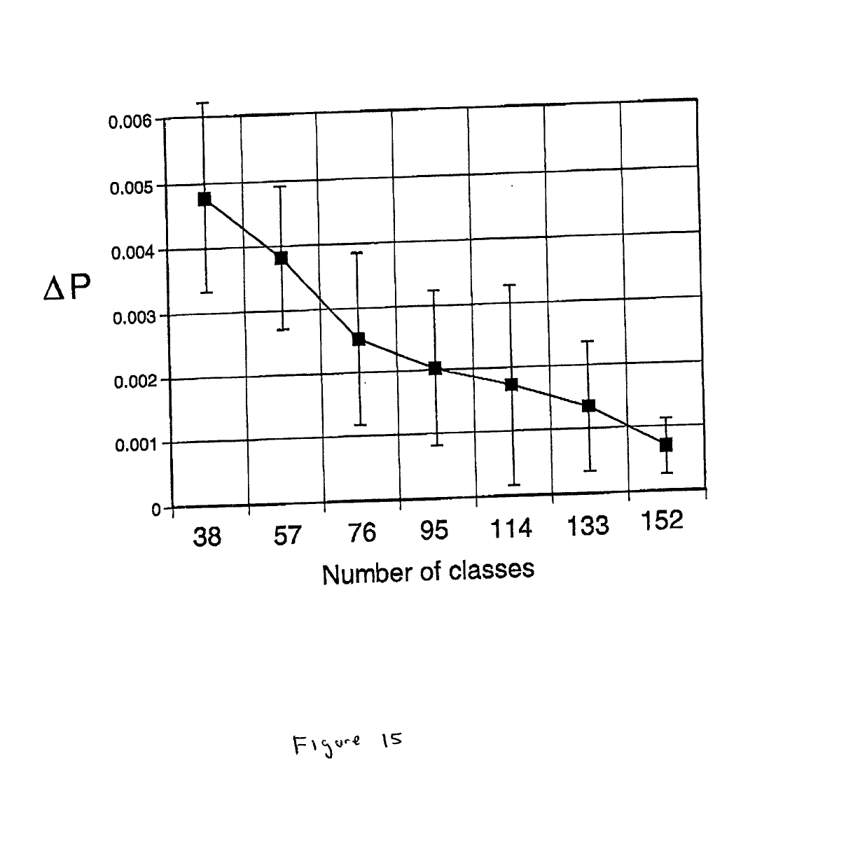

In some cases, it may be desirable to identify regions outside of the high activity region defined by the reference set. For example, one may wish to explore a region or regions of chemical space removed from areas where most active compounds have already been found. If continuing research in the active region fails to uncover new hits or leads, the void region of chemical space may provide important discoveries. Note also that sometimes one will wish to explore a subregion of the active region, when the subregion is known to have a specialized activity such as a negative charge or a large number of representative pharmacophores. FIGS. 14A and 14B present detailed maps showing important subregions within a larger region of high pharmacological activity.

Note that pharmacophore fingerprints may be used directly in library design. The Tanimoto coefficient is a convenient method for measuring the similarity between the pharmacophore fingerprints of two molecules. Briefly, the Tanimoto coefficient is defined as N_{1&2}/(N₁+N₂−N_{1&2}) where N₁ is the number of bits set in bitstring 1, N₂ is the number of bits set in bitstring 2 and N_{1&2} is the number of bits set in the bitstrings produced by a Boolean AND operation on bitstrings 1 and 2. Thus, N_{1&2} represents the number of bits that bitstrings 1 and 2 have in common.

The Tanimoto coefficient between a candidate for a library and a known biologically active molecule can give a rough or first pass indication of the candidate's potential value. Note that compounds having apparent structural dissimilarity may have similar biological activity should their pharmacophore fingerprints overlap significantly. Thus, pharmacophore fingerprints can identify obscured structural similarity between compounds. A simple comparison of Tanimoto coefficients may provide a mechanism for associating investigation set compounds with a region of high activity. A sufficiently high Tanimoto coefficient between an arbitrary member of the investigation set and any member of the reference set may indicate that the member of the investigation set should be included in a library.

As previously mentioned, a reference set of compounds should be carefully chosen in the initial development of a library. Generally, a reference set member may be any compound that has been synthesized and has a defined activity. Preferably, a reference set member is a compound known to have the activity of interest. Even more preferably, the reference set members should be structurally diverse but strongly exhibit the activity of interest.

Broadly speaking, the defined activity of the reference set can be any activity that is exhibited by a collection of chemical compounds or materials. For example, activities such as pharmacological activity, superconductivity, chromatographic mobility and fragrance or aroma can be a defined activity exhibited by a reference set that is within the context of the instant invention. Still other activities might include herbicidal properties, conventional conductivity, catalytic properties, dielectric properties, luminescence, magnetostrictive properties, ferroelectric properties, and the like. Note that members of a reference set having “biological activity” may possess drug properties unrelated to binding to a biological target such as absorption, distribution, metabolism and excretion that are defined activities within the scope of the current invention. A reference set for a primary library will typically exhibit multiple activities. The above enumeration of reference set activities is not meant to restrict the scope of the invention in any fashion.

Note that the methods of this invention are not limited to creation of primary libraries.

They may also be applied to create more constrained intermediate libraries of compounds active against a number of structurally related targets and even focused libraries.

When one wishes to design a primary library of potential pharmaceutical compounds, the reference set may include members that bind to a number of targets, which are usually biological targets (e.g., receptors and enzymes). In this particular situation, the overall region of a defined activity in chemical structure space will span multiple therapeutic activities.

2. An Example of a Pharmacologically Active Reference Set

In a preferred approach to identifying a region of pharmacological activity, the reference set comprises a significant number of known pharmacologically active compounds.

More preferably, the reference set is the newest version of the MDL Drug Data Report (MDDR), a database of known pharmacologically active compounds. The database is available from MDL Information Systems Inc., 14600 Catalina St., San Leandro, Calif. 94577. Presently, the newest version of the MDDR is version 98.1. Even more preferably, the reference set is a subset of the MDDR. In one embodiment, the reference set is a subset of the MDDR, version 98.1. The unfiltered reference set may be limited to a more refined activity such as psychotropic or vasodilator activity.

In a preferred embodiment, a specific subset of a large compound database may be used as a reference set in the procedure described in FIG. 1. Whether a subset is used depends upon how closely the database compounds, collectively, represent the desired range of activities to be represented in the primary library. In one specific embodiment, selection of a subset of the MDDR is described in detail with reference to FIG. 2. As illustrated, the database compounds may be reduced in size by using filtering procedures such as molecular weight ranges, atomic composition or structural homology. Subsets of compound databases can be generated using any useful criteria. Thus, the procedure outlined in FIG. 2 is only one example and is not intended to limit the scope of the current invention. Preferably, the depicted filtering process is automated using an appropriately configured digital computer, for example.

In step 201 the computer system receives a large database of chemical structures. In one preferred approach the database is the complete MDDR, version 98.1 which consists of 92,604 compounds. In step 203, small, disconnected fragments such as counterions are removed from the database organic structures. In a preferred embodiment, a program called “StripSalt” is used to remove the associated salts (S. M. Muskal et al., U.S. patent application Ser. No. 09/114, 694, filed on Jul. 13, 1998 which is herein incorporated by reference). The molecular weight of the pharmaceutically important organic portion of the molecule can be accurately calculated after removal of the salt moiety, which is important in subsequent steps of FIG. 2. Usually, the counterion of an organic molecule is not an important determinant of biological activity.

In step 205 compounds with molecular weights outside a certain range are eliminated from the database provided in step 201. In one particular embodiment, compounds with molecular weights that are less than about 200 Daltons and greater than about 700 Daltons are eliminated from the MDDR database. The great majority of important small molecule pharmaceutical compounds have molecular weights between 200 Daltons and 700 Daltons. However, for example, a subset that consists entirely of macromolecules could be easily constructed from a chemical database simply by specifying a molecular weight of greater than 5,000 Daltons.

The set of compounds from step 205 may be further limited by eliminating chemical structures on the basis of atomic composition in step 207. In one preferred approach, structures that possess atoms other than C, N, O, H, S, P, F, Cl, Br and I are eliminated from the database. Most important biologically active compounds are comprised only of these atoms. However, a subset that includes metal complexes could be formed from a database by specifying elimination of structures that lack at least one metal.

In step 209 close analogs may be eliminated from the reference set to avoid unduly biasing the reference set. A convenient computational measure of chemical similarity is the Tanimoto coefficient. The Tanimoto coefficient is used to compare binary bitstrings and provides a useful measure of similarity only when compounds are represented as binary bitstrings. Calculation of the Tanimoto coefficient using MDL 166 user keys, which are 2D fragment-based descriptors, has been described (M. J. McGregor et al., J. Chem. Inf. Comput. Sci., 1997, 37, 443 which was previously incorporated by reference). The MDL 166 keys are a binary descriptor that uses 166 2D substructural fragments that are automatically calculated for compounds in MDL databases and can be output for analysis. Thus, the MDL 166 keys are a binary fingerprint that contains two-dimensional information in 166 bits. For example, in one preferred embodiment, compounds with a threshold Tanimoto coefficient of greater than 0.8 are removed from the database. Other criteria such as different binding affinity for one receptor or different biological responses elicited by binding to the same receptor (e.g. agonist and antagonist activity) also be used to divide a compound database.

Next, the compounds provided in step 209 may be divided on the basis of biological activity in step 211. In one particular embodiment, compounds provided in step 209 can be divided into activity classes, which indicate affinity for a particular biological target such as an enzyme or receptor. Some compounds may have activity against a number of different targets and thus may belong to more than one activity class. Note that other criteria such as binding affinity, number of carbon atoms or types of functional groups can be used to divide a compound database. Thus, the original database of compounds may be divided into any possible number of classes.

Finally, in step 213 activity classes below a certain size are removed from the reference set. In a preferred embodiment, activity classes that have less than eight members were eliminated from the reference set.

The process outlined in FIG. 2 provides a relatively unbiased, smaller reference set from a larger database. A smaller reference set is more computationally efficient to use in the process of FIG. 1 and is thus preferable to a large reference set on this basis alone. The reference set provided by the procedure of FIG. 2 should be representative of the relevant activities of the larger database. In a preferred embodiment, the reference set is representative of features found in commercial drugs. However, a procedure similar to that of FIG. 2 could be used to prepare computationally efficient, unbiased reference sets from a larger database for any activity or activities.

3. Generating Pharmacophore Fingerprints

As indicated in FIG. 1, the reference set members are fingerprinted at step 103. Similarly, the investigation set members are fingerprinted at step 109. Fingerprinting provides a list of pharmacophores that represent the structure of a compound under consideration. One approach to fingerprinting involves assigning pharmacophoric types (e.g., negative charge, hydrogen bond donor, hydrophobic region, etc.) to substructures (e.g., atoms) of a compound to be fingerprinted. Then all of the energetically reasonable conformations of the current structure are identified for matching against the pharmacophore basis set. Matching is accomplished by comparing each reasonable conformation against the members of the pharmacophoric basis set. The system measures distances between pharmacophoric centers in a current conformation to generate candidate matches that may match one of the pharmacophores in the basis set. Positive matches between pharmacophoric candidates in a current conformation and a pharmacophore in the basis set are registered in the pharmacophore fingerprint for the current structure. When all identified conformations of the current structure have been compared against the basis set the pharmacophore fingerprint for the current structure is complete.

FIG. 3 is a flowchart detailing a preferred method for generating pharmacophore fingerprints. Preferably, the depicted process of assigning fingerprints is automated using an appropriately configured digital computer, for example.

Initially, at procedure 301, the computer system receives a basis set of pharmacophores. Preferably, such a basis set was previously constructed and made available for fingerprinting various compounds. Generally, the basis set will be developed to represent structures that may be relevant to a wide range of activities (e.g., estrogen receptor binding, retroviral reverse transcriptase inhibitors, etc.). Alternatively, the basis set may be specifically designed for a particular class of activities.

Each pharmacophore in the basis set has a collection of pharmacophoric centers; preferably all pharmacophores in the basis set have the same number of centers (e.g., three). Each pharmacophoric center is given a relative position and an associated pharmacophoric type. The relative positions define a spatial arrangement of chemical properties (i.e. the pharmacophoric types).

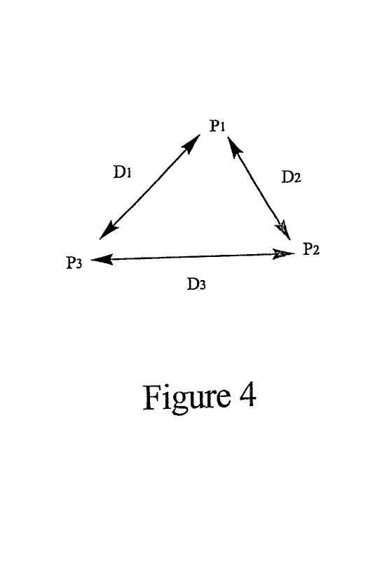

FIG. 4 depicts a three-point pharmacophore used in one type of basis set construction. Here, three pharmacophoric centers P₁, P₂ and P₃ form the vertices of a triangle. D₁, D₂ and D₃ are the distances between P₂ and P₃, P₁ and P₃ and P₁ and P₂, respectively.

The number of pharmacophore types used in basis set construction may be varied depending upon the desired application. In one preferred arrangement, the pharmacophore types available in the basis set include a hydrogen bond acceptor (A), a hydrogen bond donor (D), a group with a formal negative charge (N), a group with a formal positive charge (P), a hydrophobic group (H) and a aromatic group (R). In a more preferable embodiment, the pharmacophore types used in basis formation include the six types listed above and a default group (X) which represents a atom that is not labeled by one of the six types mentioned above.

The number and magnitude of distances that separate the pharmacophore types are also variable. The ranges should be chosen based upon distances that are expected to influence activity and represent the size of actual compounds. In a preferred embodiment, six distance ranges (D₁, D₂ and D₃) between 2.0-4.5 Å, 4.5-7.0 Å, 7.0-10.0 Å, 10.0-14.0 Å, 14.0-19.0 Å and 19.0-24.0 Å are used to form the basis set.

For a set number of centers per pharmacophore, the number of pharmacophore members in a basis set depends upon the number of available pharmacophoric types and the number of available distance ranges. Obviously, greater numbers of distance ranges and pharmacophoric types translate to greater numbers of members in a basis set. In examples described below, over 10,000 pharmacophores may be used to fingerprint compounds.

Returning to FIG. 3, after an appropriate basis set has been received at 301, the computer system next selects a current compound for fingerprinting and receives an input structure for that compound at 303. Note that many compounds will be fingerprinted in succession when a reference set or investigation set is employed. Each will be deemed the “current compound” in its turn.

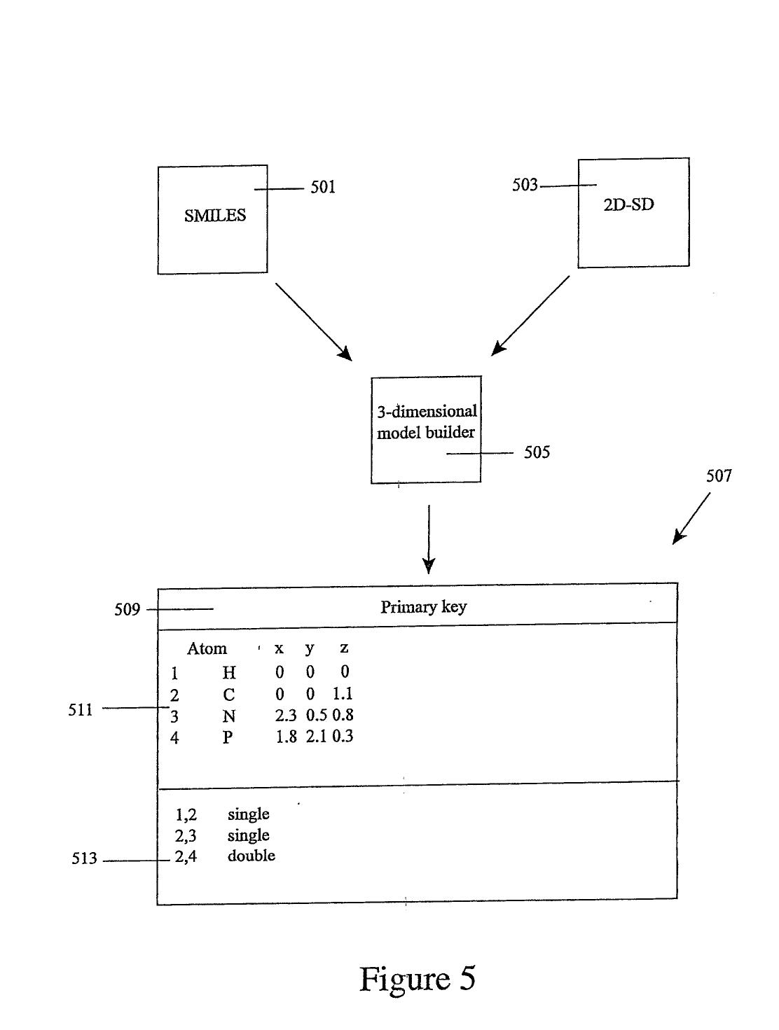

The input structure preferably specifies the relative spatial positions of the atoms of the compound and the types of bonds connecting them (ionic, covalent single, double, etc.). The atom positions should be presented in three-dimensional space. Preferably, the computer system receives the input structures of the compounds in a standardized format. The system may access the compounds from a database of such compounds. One preferred format for the input structures will be described below with reference to FIG. 5.

After the system receives the three-dimensional structure of the current compound, pharmacophore types are assigned to the atoms of the structure at 305 in FIG. 3. An atom-by-atom mapping algorithm may be used to conduct a substructure search for locations to which pharmacophore types should be assigned (D. J. Gluck, J. Chem. Doc., 1965, 5, 43 which is incorporated herein by reference). The relevant substructures typically include atoms and sometimes ring centers (e.g., aromatic centers). The pharmacophore types are assigned using heuristics that indicate which particular substructures correspond to specified pharmacophoric types. For example, an amine nitrogen may be assigned a positive charge (P), a carboxylate oxygen may be assigned a hydrogen bond acceptor (A), a phenyl group may be assigned an aromatic center (R), etc. In a preferred embodiment, an atom left unlabeled by the above procedure is assigned the X-type pharmacophore type within a higher level of procedure 305.

U.S. patent Ser. No. ______, (Docket No. 2069.1) previously incorporated herein by reference contains examples of heuristics used in a preferred embodiment of the instant invention. The heuristics define six pharmacophoric types: hydrogen bond acceptor (A), hydrogen bond donor (D), hydrophobic (H), negative charge (N), positive charge (P) and aromatic (R).

After the system assigns pharmacophoric types to the current compound, the relevant conformations of the compound are identified at 307 in FIG. 3. Preferably, this involves identifying all of the energetically reasonable conformations of the current structure. These include reasonable conformations of ring structures (e.g., the axial and equatorial conformations of cyclohexane rings), and reasonable rotational positions of various bonds. In a preferred approach, the system treats each relevant ring conformation as a separate compound possibly having its own set of rotational bond conformations. The fingerprint for such compounds is a composite of the pharmacophoric matches obtained for each ring conformation.

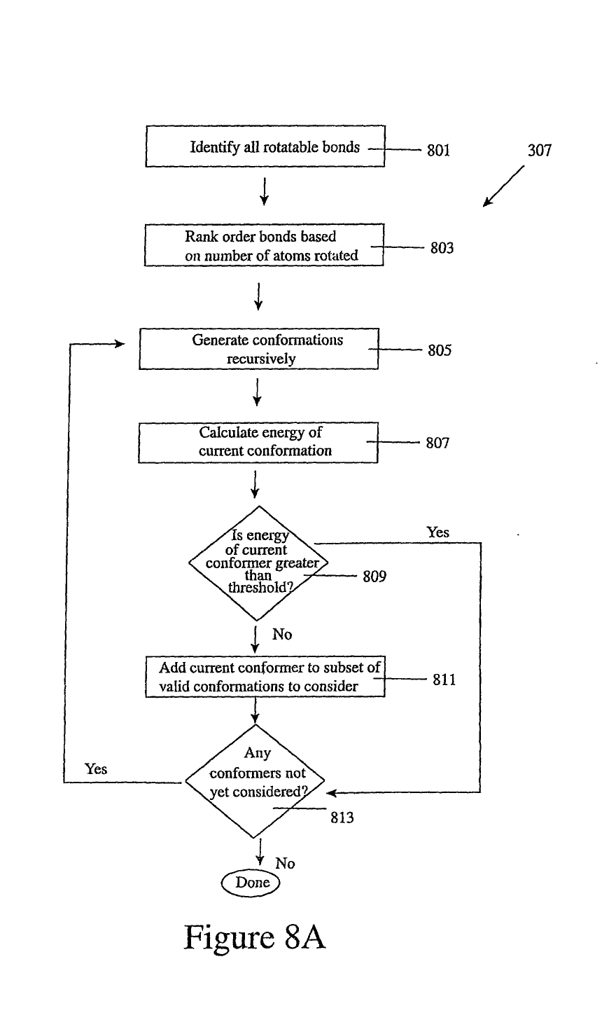

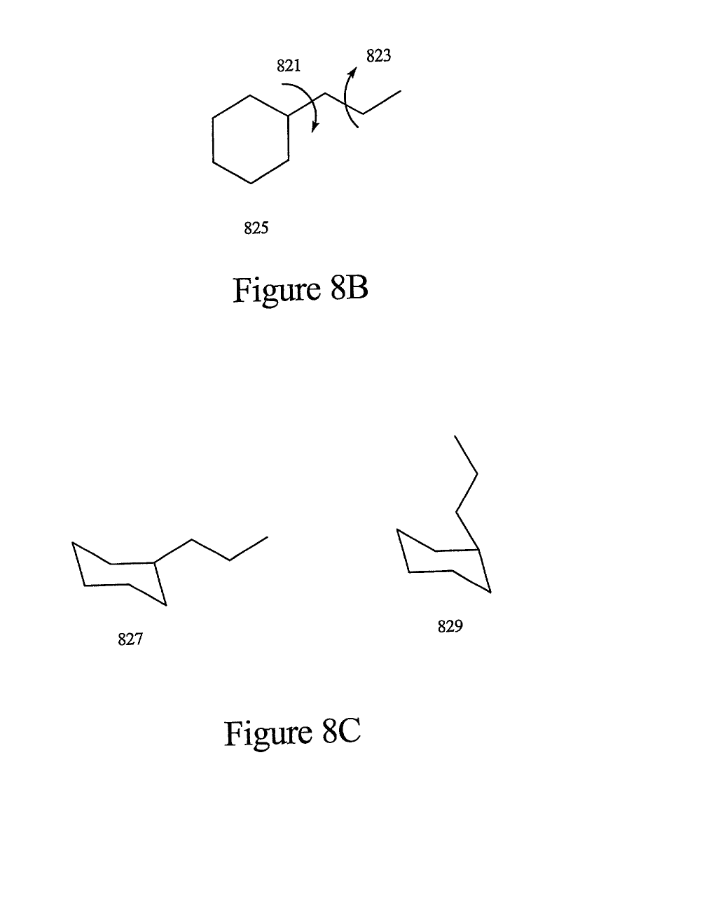

In one embodiment, all rotatable bonds of the current compound are identified. Then, the rotatable bonds are ranked based on the number of atoms of the current structure rotated. The most important bonds are ones that rotate the most number of atoms in the current structure. Then, all conformations of the current structure are generated recursively. The energy of each conformation is calculated and conformations which have energies higher than a threshold value are discarded. The remaining subset of all possible conformations is then used to generate a pharmacophore fingerprint for the current compound. To conserve computational resources, the number of possible conformations may be limited to a preset value (e.g., 1000). Preferably, the rotatable bonds that rotate the largest number of atoms are rotated first, so that if the maximum number of conformations is reached the least significant rotations are the ones that are not evaluated. Thus, in this situation, only the higher ranked conformations are considered. Otherwise, there is no significance to the order in which the possible conformers are considered. An example of a suitable conformation generation process will be presented below with respect to FIGS. 8A, 8B, and 8C.

After the computer system identifies all relevant conformations for the compound under consideration, it must consider each of them in turn. This involves selecting one conformation and matching it against the basis set, selecting another conformation and matching it against the basis set until all conformations have been matched. To represent this in FIG. 3, the system generates the three-dimensional structure of a selected current conformation at 309. Then the system matches that structure against the basis set at 311. When the matching is complete, it determines whether there are any unconsidered conformations remaining at 313. If so, process control loops back to 309 where the next conformation of the compound is selected and its three-dimensional structure is generated. This continues until all of the permissible conformers for the current structure identified at 307 have been matched against the basis set.

In a preferred embodiment, matching at 311 involves considering all possible combinations of three substructures (for three-point pharmacophores) in the current conformation. For each such combination, the system determines the associated pharmacophoric types (assigned at 305) and separation distances. This specifies a candidate that the system compares against all pharmacophores in the basis set. Any matches are stored as a contribution to the fingerprint. In the final fingerprint, the bit positions corresponding to matched basis set pharmacophores are set to 1.

After the system has considered all relevant conformers for the current compound, decision 313 is answered in the negative. At that point, process control moves to 315 where the bit-by-bit fingerprint for the current compound is completed. Generally, the fingerprint is complete only after all relevant conformers, including those depending upon alternative ring conformations, are considered.

In one embodiment the pharmacophore fingerprint for the current structure includes a binary bit string that is η bits long, where η represents the number of pharmacophores in the basis set. Each bit position represents one pharmacophore in the basis set. In a preferred arrangement, the pharmacophore fingerprint of the current compound consists of a bitstring with 10,549 bits with each bit corresponding to a unique member of the basis set pharmacophores.

The bit position may contain a 1 that indicates that the corresponding basis set pharmacophore is present in at least one conformation of the current compound. Alternatively, the bit position may contain a zero which means that the corresponding basis set pharmacophore is absent from any energetically reasonable conformations of the current compound. The output from 315 may include, in addition to a complete pharmacophore fingerprint for the current structure, a “compound identifier” in a specified data field that is a label that keeps track of the current compound.

The fingerprint can assume other formats. In the format just described, a given pharmacophore is represented by a single bit and is given a value of 1 no matter how many times that pharmacophore occurs in the compound. Note that it is entirely possible that a given pharmacophore from the basis set may be appear multiple times in a compound. In an alternative format, the number of times a pharmacophore occurs is specified in the fingerprint. Other formats will be apparent to those of skill in the art.

To conserve storage space, the computer system may compact the pharmacophore fingerprint at 317. For example, if a 32 bit computer is used 32 bits in the fingerprint bit string are represented as one integer in computer memory. Thus a bit string that consists of 10,549 bits is compacted into 330 integers in computer memory. Alternatively, if a 64 bit computer is used 64 bits in the bitstring are compacted into one integer. Thus a bit string that consists of 10,549 bits is compacted into 165 integers in computer memory. The pharmacophore fingerprint can be easily unpacked into one integer or floating point number per bit if necessary for calculations. Note that unpacking may be unnecessary for some calculations. For example, the Tanimoto coefficient can be calculated using bitwise operators in a conventional programming language.

After the system generates and stores the current compound's fingerprint in an appropriate format, it determines whether any compounds remain to be considered. See decision branch point 319. Remember that a reference set or investigation set may contain many different compounds, each of which should be fingerprinted. If the answer at 319 is yes then the program loops back to 303 to receive an input structure for the next compound to be fingerprinted (the new “current compound”). If the answer is no then a pharmacophore fingerprint has been constructed for every member of the reference set or investigation set and the process is complete.

As indicated above, a fingerprint may contain indicia of each pharmacophore in a basis set. In FIG. 3, the basis set is made available at 301. The system uses the basis set during matching at 311. In the above discussion, the pharmacophores of the basis set include three points. In other words, the pharmacophores usually define triangles and occasionally define lines. It is, of course, possible that pharmacophores of the basis set may include two, four, five, or six centers. A two-point pharmacophore must be one-dimensional and a three-point pharmacophore may be one- or two-dimensional. Pharmacophores having more centers may be one, two, or three-dimensional.

Each pharmacophoric center in a pharmacophore is assigned a pharmacophoric type. Examples of pharmacophoric types include aromatic centers (R), hydrogen bond acceptors (A), hydrogen bond donors (D), centers with a negative charge (N), centers with a positive charge (P), and hydrophobic centers (H). In a preferred embodiment, a default type (X) may be used for any atom that is not labeled with any other designated type. In an especially preferred embodiment, the pharmacophoric types include the above seven types.

In a specific embodiment, the pharmacophoric centers are separated by six distance ranges (for D₁, D₂ and D₃ in FIG. 4) that are between 2.0-4.5 Å, 4.5-7.0 Å, 7.0-10.0 Å, 10.0-14.0 Å, 14.0-19.0 Å and 19.0-24.0 Å. It should be borne in mind that the number of pharmacophore types and the number and value of distance ranges used in forming a basis set can be easily varied.

A diverse basis set of pharmacophores may be generated by forming all possible combinations of pharmacophore types and distances. In a preferred arrangement, two additional constraints reduce the size of a basis set comprised of three-point pharmacophores. The triangle rule eliminates geometrically impossible three-point pharmacophores. Referring now to FIG. 4, if the length of a side of the triangle defining the three-point pharmacophore, exceeds the sum of the lengths of the other two sides that particular pharmacophore is removed from the basis set. Second, three-point pharmacophores that are related by symmetry group operations to three-point pharmacophores already present in the basis set are eliminated from the basis set.

In one example, a basis set includes 10,549 three-point pharmacophores with seven distinct pharmacophore types and six distinct distance ranges after application of the two constraints discussed above. Alternatively, a basis set may include 6,726 three-point pharmacophores with six pharmacophoric types separated by six possible distance ranges after application of the two constraints discussed above.

As mentioned, the basis set should be sufficiently large to define most structures relevant to activity. For most situations, the basis set preferably includes at least about 5,000 members and more preferably includes at least about 10,000 members.

The structural representation of a current compound used for fingerprinting must be susceptible to comparison with the pharmacophore basis set. It must indicate when a match occurs against a pharmacophore. Because pharmacophores are defined by a group of pharmacophore types separated by defined distances, a compound's structural representation should indicate pharmacophore types and the separation distances.

Conveniently, compounds may be represented in a conventional format such as SMILES, 2D-SD, etc. Such formats represent compounds as lists of atoms connected by specified bonds. To be available for matching against pharmacophores, the atoms of the compounds must first be represented in three-dimensional space. The compounds may then be used in the process of FIG. 3 (operation 303).

One approach to generating a three-dimensional structure useful in the process of FIG. 3 is illustrated in FIG. 5. As illustrated, the current compound is provided in a SMILES format (501), a 2D-SD format (503) or any other suitable two-dimensional structure file. This representation is provided to a three-dimensional model builder (505) that converts the atom and bond information contained in the input file to a three-dimensional representation 507. Model builder 505 then outputs three-dimensional representation 507 as illustrated.

Model builder 505 may be any module that can generate three-dimensional coordinates of atoms in a compound. One preferred example of a model builder is the “Corina” software program available from Oxford Molecular, Ltd., Oxford, England. (J. Gasteiger et al., Tetrahedron Comp. Method, 1990, 3, 547 which is incorporated herein by reference). This program runs in batch mode, accepts a variety of standard molecule formats, and has been observed to generate good quality structures (J. Sadowski et al., J. Chem. Inf. Comput. Sci., 1994, 34, 1000 which is incorporated herein by reference).

Shown in FIG. 5 is a representative data structure presenting a three-dimensional structural representation that may be employed as input at 303 in FIG. 3. The representation includes a primary key 509 that uniquely identifies the current compound. Note that the current compound may have been selected from a database of compounds, and that each compound in the database is uniquely identified by a primary key. The data structure also includes an atom block 511 that uniquely labels each atom in the compound by number. It also specifies the associated element and three-dimensional position of the element. For example, the atom block contains information that atom 1 is hydrogen, atom 2 is carbon, atom 3 is nitrogen and atom 4 is phosphorus. The data structure specifies the three-dimensional position of each atom by the x, y, and z Cartesian coordinates. Data structure 507 also includes a bond block 513 that contains the connectivity between the atoms and the bond order. In the example shown, atom 1 is connected to atom 2 and is a single bond, atom 2 is connected to atom 3 and is a single bond and atom 2 is connected to atom 4 and is a double bond.

The three-dimensional atomic representation of the current compound must be converted to a three-dimensional pharmacophoric representation (305 of FIG. 3). This may be accomplished through the use of a heuristics that consider the elements making up the compound and their environments within the compound. From these considerations, pharmacophoric types are assigned to substructures (e.g., atoms or aromatic centers) positioned in the three-dimensional space occupied by the compound. A complete listing of sample heuristics that may be used in procedure 305 of FIG. 3 has been presented (M. J. McGregor and S. M. Muskal, “PHARMACOPHORE FINGERPRINTING IN QSAR” U.S. patent Ser. No. ______ (docket no. 2069.1) which was previously incorporated by reference). In this sample (and most of the discussion presented herein), the only structures considered are those that consist entirely of atoms from the following list: carbon, nitrogen, oxygen, hydrogen, sulfur, phosphorus, fluorine, chlorine, bromine and iodine. The invention is not, of course, limited to such compounds.

In one example of an assignment of a pharmacophoric type to a substructure, a carboxylate group oxygen is assigned a negative charge (N), a hydrogen bond acceptor (A), an aliphatic amine is assigned a positive charge (P), and a hydroxyl group is assigned both a hydrogen bond donor (D) and acceptor (A). Significantly, hydrogen atoms are not assigned a pharmacophoric type. In one heuristic, the hydrophobic pharmacophore type is assigned to a carbon, chlorine, bromine, or iodine atom that is more than two bonds removed from a nitrogen, oxygen, phosphorus, or mercaptan functionality.

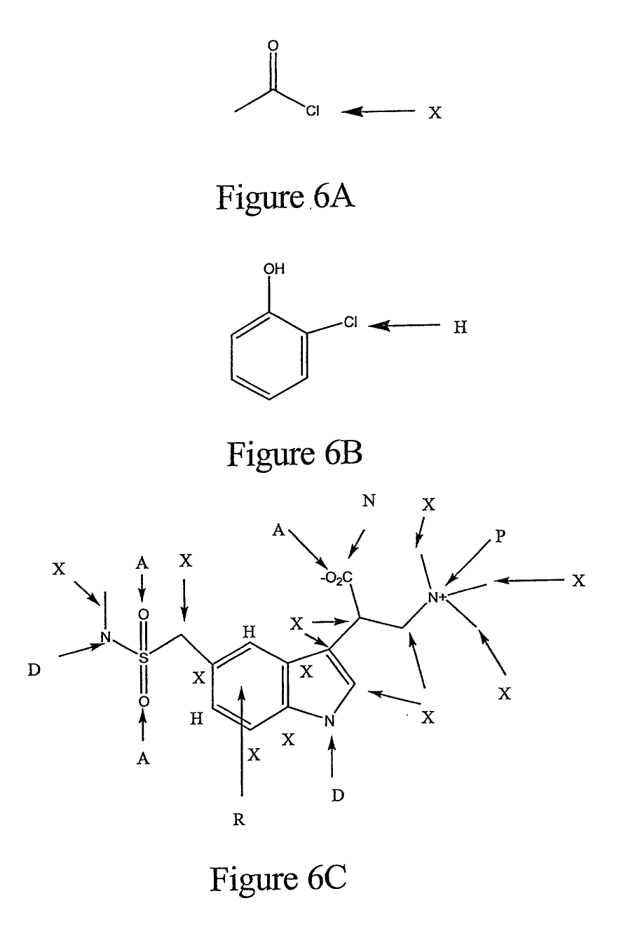

FIGS. 6A, 6B and 6C illustrate pharmacophore type assignment to atoms. FIG. 6A show a simple acyl chloride. The chlorine atom is assigned the default pharmacophoric type (X) because it cannot be described by any of the other six pharmacophore types. Note that it is within two bonds of an oxygen atom, so it can not properly be categorized as a hydrophobic (given the above heuristic). In contrast, the chlorine atom of ortho chlorophenol shown in FIG. 6B is assigned a hydrophobic pharmacophoric type (H) because more than two bonds separate it from the phenolic hydroxyl group.

FIG. 6C illustrates an analogue of sumatriptan that contains each of the seven pharmacophoric types used in a preferred embodiment. Starting from the left of the structure and moving to the right, the methyl group carbon attached to the nitrogen is assigned a default pharmacophoric type (X). This assignment was made because the carbon does not qualify as a hydrogen bond donor or acceptor, a positive or negative charge center, a hydrophobic site (it is bonded to a nitrogen atom), or an aromatic group. The nitrogen atom bonded to the methyl carbon is assigned a hydrogen bond donor (D) pharmacophoric type. The sulfonyl oxygens are assigned hydrogen bond acceptor (A) pharmacophoric types while the sulfur atom is assigned a default (X) pharmacophoric type. The methylene group between the benzene ring and the sulfonamide is assigned a default (X) pharmacophoric type. The benzene ring is assigned an aromatic (R) pharmacophoric type. The locus of the R assignment is the centroid of the benzene ring. The substituted benzene carbon is assigned a default (X) pharmacophoric type while the adjacent aromatic carbons may are assigned a hydrophobic (H) pharmacophoric type. The remaining benzene carbons are all assigned a default (X) pharmacophoric type. The indole nitrogen is assigned a donor (D) pharmacophoric type while the indole carbon adjacent to the indole nitrogen is assigned a default (X) pharmacophoric type. The other indole carbon is and the methylene group adjacent to the indole ring are also assigned a default (X) pharmacophoric type. The carboxylate functionality is assigned both a negative (N) and an acceptor (A) pharmacophoric type. Significantly, the carboxyl group is an example of a pharmacophoric center that can be represented by two different pharmacophore types. Finally, on the right hand side of the molecule, the methylene group and the methyl groups adjacent to the fully alkylated amine are assigned a default (X) pharmacophoric type while the amine nitrogen is assigned a positive (P) pharmacophoric type.

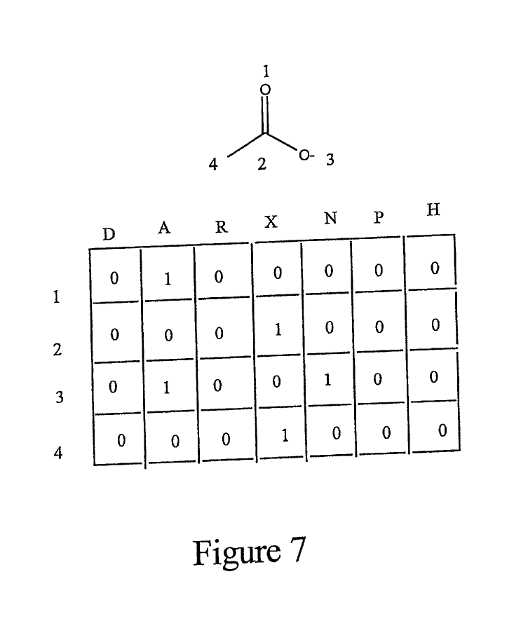

To facilitate matching (311 of FIG. 3), the system creates a data structure representing the current compound with pharmacophoric types specified. FIG. 7 illustrates an example of such a data structure 703 for the anion of acetic acid 705. Generally, the classification of atoms into different pharmacophore types are contained in a n x (p array where η represents the number of atoms other than hydrogen atoms while φ represents the number of pharmacophore types. Thus, in this particular example, the array is 4×7 corresponding to the number of atoms other than hydrogen atoms and the number of pharmacophoric types respectively. For each array cell, the corresponding atom either is or is not assigned the corresponding pharmacophoric type. In this example, the presence of a 1 indicates that the atom in question can be represented by particular pharmacophore type while a 0 indicates that it cannot. Thus, atom 1, a carbonyl oxygen, has a 1 in the acceptor (A) pharmacophoric type columns. All other columns are set to 0 for atom 1. Atom 2, the carbonyl carbon, has a 1 in the default (X) pharmacophoric type column. Atom 3, a carboxylate oxygen, has 1 in the acceptor (A) and the negative charge (N) pharmacophoric type columns. Atom 4, the methyl carbon has a 1 in the default (X) pharmacophoric type.

Some general points about pharmacophore type assignment are made below. Preferably, hydrogen atoms are not assigned pharmacophoric types. Generally, atom numbering is arbitrary. In one preferred embodiment the same atom numbering is used in pharmacophore assignment, Corina and the original input data. In another embodiment, aromatic centers are added psuedoatoms. In another preferred embodiment, bonds are either single or double bonds; partial double bonds, characteristic of resonance stabilized structures are not permitted.

As indicated in operations 307 and 309 of FIG. 3, the system generates relevant conformations for the current compound and then considers each of these separately for matching against the pharmacophoric basis set. Preferably, the system considers only those conformations that do not result in significant steric overlap. Many conformations that are severely sterically hindered do not exist or exist only for very short time durations because their internal energy is too great. Preferred methods exclude conformers with high internal energies because they do not contribute significantly to biological activity.

FIG. 8A is a flowchart that illustrates a preferred method for generating conformation(s) of a chemical structure for pharmacophore fingerprinting utilizing a quaternion rotation algorithm (K. Shoemake, SIGGRAPH, 1985, 19, 245-254 which is incorporated herein by reference). Thus, FIG. 8A may represent operation 307 in FIG. 3.

Initially, the computer system at 801 identifies all rotatable bonds in the current structure. Well-known heuristics may be used to determine which bonds can be rotated and the angles at which they can be rotated. For example, a SP₃-SP₃ bond has 3 rotamers that differ by 120°. A SP₂-SP₂ bond has two rotamers that differ by 180°. Generally, bonds in rings are assumed to not be rotatable. A multiple ring conformation option of some three-dimensional model builders (e.g., the Corina program) provides conformational isomers of common ring compounds. These ring conformers may be used independently of one another to generate separate groups of conformers based on rotations about non-ring bonds. Each conformer from the two groups is separately matched against the basis set to form the compound's fingerprint.

Reference to FIG. 8B illustrates operation 801. FIG. 8B illustrates propyl cyclohexane, a compound where rotation around bonds 821 and 823 generates conformational isomers. These two bonds are identified in operation 801 of FIG. 8A. Further, although the bonds in the cyclohexane ring are not rotatable, the model builder preferably provides both the axial and equatorial conformational isomers of the mono-substituted cyclohexane. Redundant conformations are eliminated by identifying symmetrical fragments (e.g. phenyl etc.) and considering bonds to them to be non-rotatable.

Returning now to FIG. 8A, the system at 803 ranks the rotatable bonds based on the number of atoms rotated because rotations about bonds moving greater numbers of atoms explore a greater range of conformation space. In the example of FIG. 8B, rotation of bond 821 moves two atoms. Thus, bond 821 would be ranked over bond 823 which when rotated moves only one atom. Bonds that rotate the same number of atoms have the same rank and one of these bonds is chosen to be rotated first in an arbitrary manner.

After the system ranks all rotatable bonds, it recursively generates all possible conformations for the current structure. The generation of each new conformer is represented by operation 805 in FIG. 8A. Note that branches in the recursion are defined by individual bonds in the compound, with higher branches corresponding to higher ranked bonds. The total number of conformations of propyl cyclohexane is 18 (i.e., 3×3×2). First are the rotational isomers of the cyclohexane ring 827 and 829 where the propyl group is oriented axially (827) and equatorially (829). Rotation around bond 821 provides three rotamers. Similarly, rotation around bond 823 yields three additional rotamers (per original rotamer on bond 821).

Each time a given conformer in the recursion is generated at 805, the system must determine whether to save that conformer for pharmacophoric matching or dispose of it as irrelevant. The system accomplishes this goal via procedures 807, 809, and 811 FIG. 8A. At 807, the system calculates the energy of the current conformation. A simple energy function (such as the Lennard-Jones potential of the AMBER force field) may be used to calculate the energy of the rotamer. Basically, this involves summing the attractive and repulsive forces between atom pairs in the current conformation. (S. J. Weiner et al., J. Am. Chem. Soc., 1984, 106, 765 which is incorporated herein by reference).

After calculating the energy of the current conformation, the system compares at 809 the energy of that conformation with a specified threshold energy value. Generally, the threshold value is set at a large value. In one specific embodiment, the threshold energy is about 100.0 kcal/mole. If the energy of the conformer is greater than the threshold value the conformation is eliminated thus removing sterically unfavorable rotational conformers of the current compound. If the energy of the conformer is less than the threshold value then it is added to the subset of conformers identified for further processing as shown in operation 811 of FIG. 8A. More specifically, this subset represents those rotational conformers that are to be matched against the pharmacophore basis set in operation 311 of FIG. 3 and thus contribute to the pharmacophore fingerprint of the current compound.

After the current conformation has been accepted or discarded, the system determines (813) whether any remaining conformers remain to be considered. This involves determining whether all conformers on the recursion tree have been considered. If not, process control returns to 805 where the system generates the next conformer on the recursion tree. That conformer's energy is then calculated and compared to the threshold as described above. If the conformer's energy is below the threshold, it is added to the subset of conformers for pharmacophoric matching. Each conformer is considered in this manner until the last one is encountered. At that point, operation 813 is answered in the negative and the process is complete. Note that in some embodiments, the last recursion proceeds to only a specified number of iterations (e.g., 1000). The maximum number of conformers evaluated is user defined and can thus be easily varied. Thus, not all conformers have their energies considered. This cut off is employed to save computational resources on very flexible compounds, where many conformations have already been identified for matching.

4. Identifying “High Activity” Regions of Chemical Space

Association of pharmacophore fingerprints of a reference set to a defined activity or multiple activities was referenced as operation 105 in the process flow of FIG. 1. As mentioned, association may be generated with any suitable technique. A preferred technique is Principal Component Analysis (P. Geladi, Anal. Chim. Acta, 1986, 185, 1, which is herein incorporated by reference). Alternatively, methods such as multiple regression techniques, partial least squares, back-propagation neural networks and genetic algorithms can also be used to associate pharmacophore fingerprints to a defined activity.

Operation 105 in the process flow of FIG. 1 requires Principal Component Analysis of the reference set. As previously suggested, the dimensionality of the pharmacophore fingerprint may be defined by the number of pharmacophores in the basis set. In a preferred arrangement, the pharmacophore fingerprint has about 10,549 different dimensions with each dimension corresponding to a different pharmacophore in the basis set. Thus, in the bit sequence representation of pharmacophore fingerprints each individual bit corresponds to an axis for a representation of chemical space. The chemical space defined by the pharmacophore fingerprints of this particular embodiment consists of 10,549 dimensions. Each compound of the reference set has a position in chemical space that is represented by its pharmacophore fingerprint bit values.

Association represents an attempt to find a relationship between two groups of variables. One set of variables is the dependent set of variables and is a function of the independent set of variables. In this invention, the dependent variables are usually one or more activity classes and the independent variables are the pharmacophore fingerprints of the reference set members (e.g., a subset of the MDDR). Using the reference set created by the process of FIG. 2, there are 152 dependent variables (corresponding to the activity classes) and 10,549 independent variables (corresponding to the dimensionality of the pharmacophore fingerprint).

A linear regression equation relates independent and dependent variables (Y=XB+e where Y is the dependent variable represented by a matrix (i.e. activity of the reference set members), X is the independent variable represented by a matrix (i.e. pharmacophore fingerprints), B is the regression coefficient represented by a matrix, and e is the residual).

Principal Component Analysis allows matrix X to be written as the sum of the outer product of two vectors, a score vector T and a loading vector P as shown in FIG. 11. In one particular embodiment, X represents the pharmacophore fingerprints and T represents the new coordinates in reduced dimensional space. The loading vector P can be applied to new fingerprints to transform them to the same reduced dimensional space. Thus, Principal Component Analysis reduces the dimensionality of matrix X to a lower dimensional space that may be pictorially represented. As mentioned previously, the pharmacophore fingerprints represent the independent variables in the analysis. The activities of the reference set member are the dependent variables. In one embodiment, the biological activity will be either 1.0 or 0.0 when the reference set consists of members that are classified as either active or inactive respectively. In a preferred embodiment, when a subset of the MDDR is the reference set, the biological activity is a binary value.

In a preferred arrangement, a nonlinear iterative partial least squares (NIPALS) algorithm, which is conveniently implemented on a digital computer, can be used to calculate the score vector T and the loading vector P (P. Geladi, Anal. Chim. Acta, 1986, 185, 1, which has been previously incorporated by reference). NIPALS does not calculate all of the principal components at once. Instead, each component is calculated by an iterative procedure that continues until the NIPALS algorithm converges.

In another embodiment, the eigenvector/eigenvalue equations can be solved to provide the principal components of matrix X. The NIPALS algorithm and the eigenvector equations should provide the same answer.

In a preferred embodiment, Principal Component Analysis of the reference set in step 105 transforms a chemical space that includes dimensions for the pharmacophore basis set to a chemical space that includes dimensions for principal components. For example, a chemical space of 10,549 dimensions can be reduced to a chemical space of between about two and ten dimensions.

Furthermore, transformation of a data matrix of the reference set to a small number of principal components can allow, in one preferred arrangement for graphical representation of the compounds of the reference set in a chemical space with the principle components as the dimension axes. In one embodiment, the principal components 1 and 2 are the dimension axes. FIG. 13A is an example of the above representation. In another embodiment, shown in FIG. 13B, the principal components 2 and 3 are the dimension axes. Four or more principal components may be used as dimension axes but pictorial representation of these chemical spaces may be difficult.

The process of step 111 involves transforming the pharmacophore fingerprints of the investigation set to the representation of chemical space obtained after operation 105. In a preferred embodiment, the pharmacophore fingerprints of the investigation set are transformed from a first representation of chemical space that includes the pharmacophore basis set as dimensions to a second representation of chemical space that includes the principal components as dimensions. The transformation of the pharmacophore fingerprints of the investigation set to the principal component space of 105 may be performed using the loadings matrix P calculated at 105.

Thus, transformation of the investigation set fingerprints to a simpler set of principal component coordinates can allow, in one preferred arrangement, for graphical representation of the compounds of the investigation set in the chemical space of the reference set with the principle components as the dimension axes. Preferably, the first two or the first three principal components are used as the dimension axes.

5. Calculating Overlap or Molecular Diversity of Investigation Set Subsets With High Activity Regions of Chemical Space

The process of step 113 is concerned with calculating overlap or the molecular diversity of subsets of the investigation set with high activity regions of chemical space. One simple procedure is selecting a subset of the investigation set that has substantial overlap with the reference set. This subset may identify the compounds comprising a new primary or constrained library. Another simple procedure is selecting from the “active” subset of the investigation set a subset based on molecular diversity criteria. If the investigation set is large or particularly diverse, it may be desirable to use more sophisticated procedures to select members of a library. As previously mentioned, a number of selection procedures may be used to identify suitable subsets of the investigation set.

In a preferred embodiment, a genetic algorithm is used to select a subset of the investigation set. Briefly, genetic algorithms are a subset of evolutionary algorithms which are algorithms inspired by the mechanisms observed in natural selection. Thus, genetic algorithms use features such as reproduction, random variation, competition and selection, which are prominent in evolution to provide a superior solution over time. The steps of a classic genetic algorithm include: (1) randomly initialize a starting population of N members; (2) assign each member a fitness score using a fitness function; (3) select a pair of parents for reproduction; (4) generate offspring using crossover and/or mutation; (5) assign each offspring a fitness score using a fitness function; (6) replace least fit members of population by the offspring if latter are superior in fitness; (7) go to point 3 until termination or convergence.

FIG. 9 represents one embodiment of the current invention that uses a genetic algorithm to select a subset or subsets of the investigation set that have substantial overlap with the reference set or are selected on the basis of molecular diversity. The process flow of FIG. 9 begins at 901 where cubic cells for a principal component representation of chemical space are defined. The division of chemical space into cells is arbitrary and may be varied as experimentally necessary. The number of dimensions of the cells generally corresponds to the dimensionality of the chemical space used to perform this analysis. Within these cells, the relative numbers of molecules of both the reference set and the investigation set may be counted. In the depicted embodiment, the investigation set is divided (typically randomly) into a number of subsets, each of which represents or is an attempted solution of the problem at hand at 903 in the process flow of FIG. 9. In one specific embodiment the current subsets may be randomly selected members of a combinatorial library. The population of the current subsets can be random or biased as desired. This step corresponds to initializing a starting population in a generic genetic algorithm.

At step 905 a function that determines, for example percentage overlap or measures molecular diversity, of the current subsets of the investigation set with the reference set is calculated. In this embodiment, the percentage overlap or measure of molecular diversity is the fitness function used to evaluate the subsets of the investigation set. Procedures that calculate percentage overlap or provide a measure of molecular diversity are well known to those of skill in the art (M. Snarey et al., J. Mol. Graphics Modeling, 1998, 15(6), 372 which is herein incorporated by reference). In one embodiment, the relative numbers of members from the investigation and reference sets are counted in each cell. As the cellular ratio of these numbers (investigation:reference) averaged over all cells approaches the ratio of total investigation set members to total reference set members, the value of the function increases.

A current subset, which is randomly selected, is now randomly mutated at step 907. In one embodiment, when the current subset is derived from a combinatorial library, randomly selected monomer units present in the subset may be exchanged with randomly selected monomers not found in the subset. In other situations mechanisms such as crossover may be used to mutate the current subset. Then at 909 the function is calculated using the mutated subset. Generally, the same function used in 905 is used at 909.

Process control passes to step 911 after calculation of the fitness function at 909. Decision point 911 determines whether the mutation made at 907 should be accepted. In one particular embodiment a Metropolis function is used to decide whether the mutation is accepted or rejected (W. H. Press et al., Numerical recipes in C, page 344, Cambridge University Press, 1988 which is herein incorporated by reference). A Metropolis function accepts a mutation that improves the function value. When the function is not improved, mutation is accepted with a probability that is dependent on the difference between the current function and the function at the previous mutation. The probability of accepting a mutation that does not improve the figure is reduced as the algorithm proceeds. Various methods of evaluating the mutation are known to one of skill in the art.

When mutation of the current subset is accepted at step 911, process control returns to 907. In this situation, the mutated subset becomes the current subset, which is again mutated at 907. Alternatively, when the mutation is rejected at 911 the system moves to 913.

The current subsets are checked for convergence at the decision point 913 in FIG. 9. Convergence can be evaluated by a number of different procedures, which are well known to one skilled in the art. For example, a threshold value of percentage overlap or molecular diversity can be used to evaluate convergence at decision point 913. Alternatively, the amount of improvement in overlap or molecular diversity, from one iteration to the next iteration can be monitored and when it reaches a sufficiently low value, the convergence criteria have been met. In one particular embodiment, convergence is reached if no improvement of the function is achieved after a certain number of attempts.

Preferably, decision point 913 evaluates whether the function is still improving. If the decision is yes (convergence has been attained), the process is completed and system selects the current subset as the “best” subset. Preferably, that subset will have the best possible value of the function.

If the decision at 913 is negative, process control loops back to step 907 where the current subset is again randomly mutated. Importantly, in this situation the current subset is identical to the current subset in the previous iteration since the mutation of the previous iteration was rejected. Enough iterations of the process represented by steps 907, 909, 911 and 913 will usually provide a subset of the investigation set with maximal value for the calculated function. This particular subset of the investigation set may constitute a primary library.

The primary library will ideally reflect the properties of the reference set which served as a template for its construction. For example, if the MDDR was used as the reference set, the primary library should be effective against at least the same biological targets. Thus, in principle the primary library, could provide new lead compounds against known biological targets. Alternatively, the primary library can be used to screen new biological targets whose ligands and structure are unknown. Since the compounds contained in the MDDR have a common mode of activity against known biological targets it may be expected that a primary library constructed using the method of the present invention will be active against new biological targets. Furthermore, the principle of primary library design is also particularly applicable to the evaluation and design of combinatorial libraries.

6. Computer Systems for Implementing the Invention

Generally, embodiments of the present invention employ various process steps involving data stored in or transferred through one or more computer systems. Embodiments of the present invention also relate to an apparatus for performing these operations. This apparatus may be specially constructed for the required purposes, or it may be a general-purpose computer selectively activated or reconfigured by a computer program and/or data structure stored in the computer. The processes presented herein are not inherently related to any particular computer or other apparatus. In particular, various general-purpose machines may be used with programs written in accordance with the teachings herein, or it may be more convenient to construct a more specialized apparatus to perform the required method steps. The required structure for a variety of these machines will appear from the description given below.

In addition, embodiments of the present invention further relate to computer readable media or computer program products that include program instructions and/or data (including data structures) for performing various computer-implemented operations. The media and program instructions may be those specially designed and constructed for the purposes of the present invention, or they may be of the kind well known and available to those having skill in the computer software arts. Examples of computer-readable media include, but are not limited to, magnetic media such as hard disks, floppy disks, and magnetic tape; optical media such as CD-ROM disks; magneto-optical media such as floptical disks; and hardware devices that are specially configured to store and perform program instructions, such as read-only memory devices (ROM) and random access memory (RAM). The data and program instructions of this invention may also be embodied on a carrier wave or other transport medium. Examples of program instructions include both machine code, such as produced by a compiler, and files containing higher level code that may be executed by the computer using an interpreter.

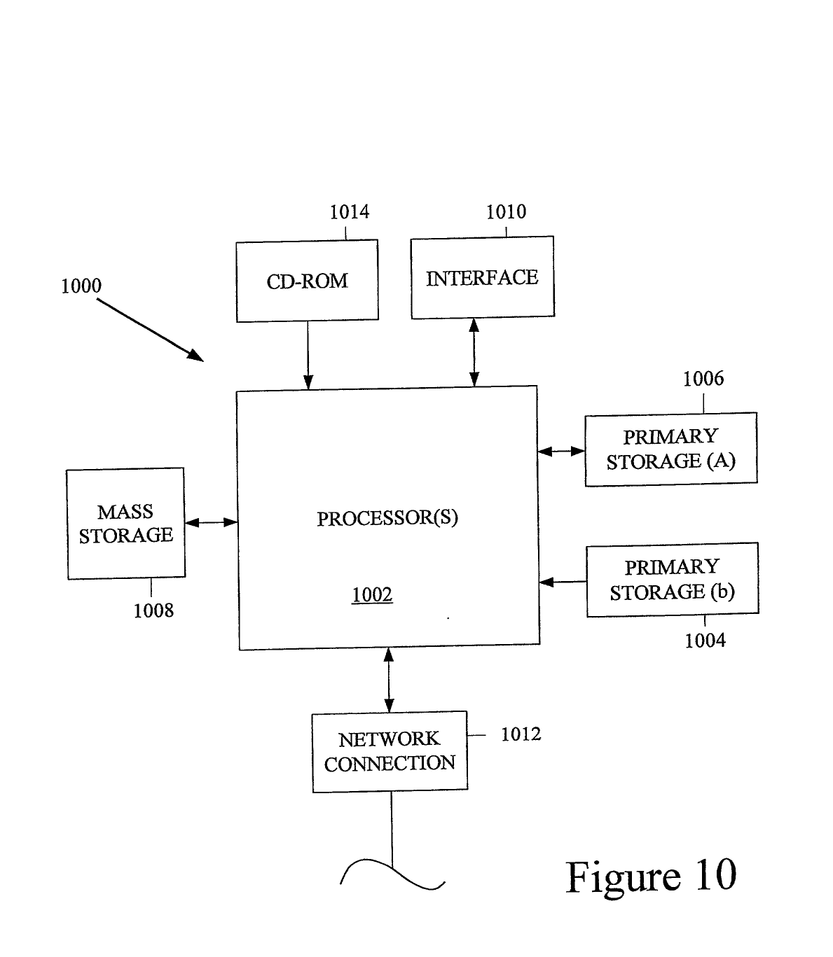

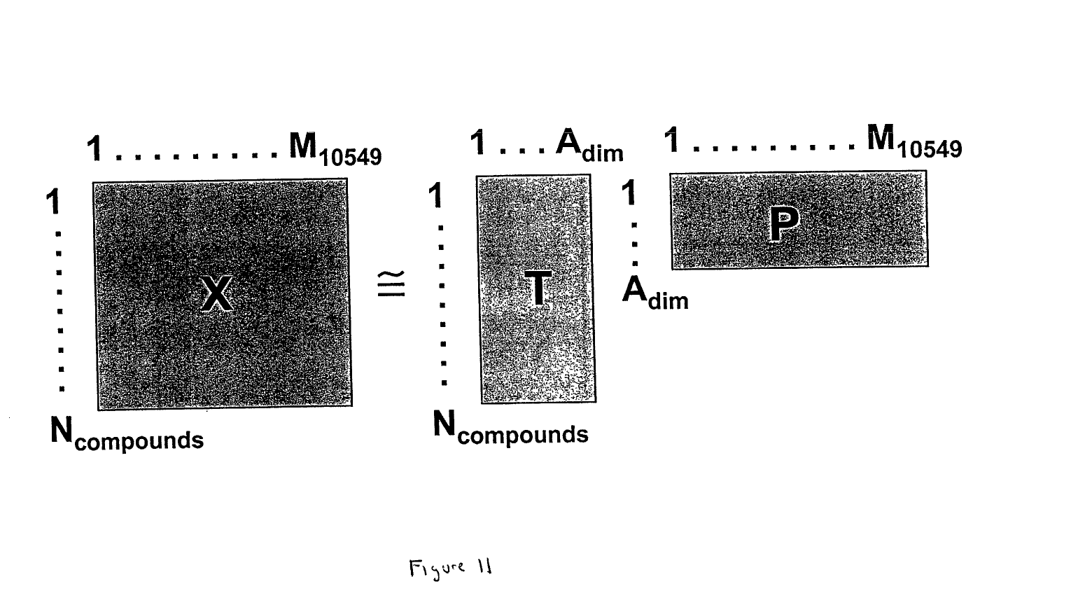

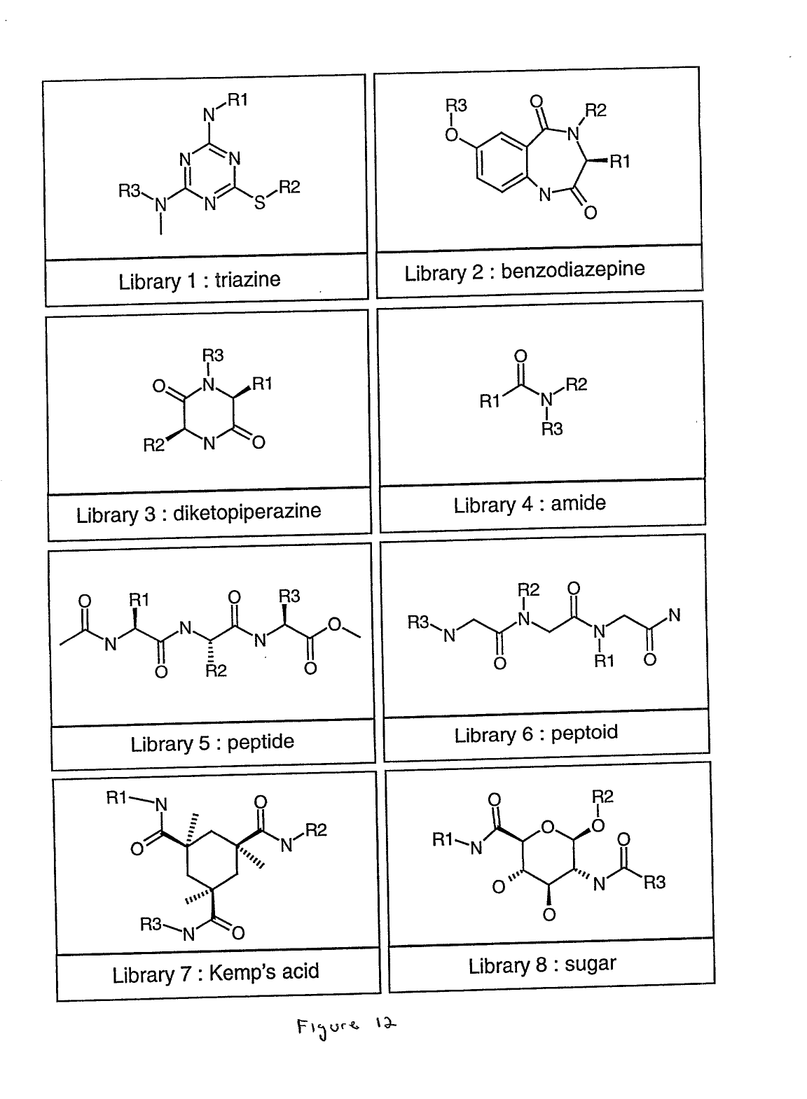

FIG. 10 illustrates a typical computer system in accordance with an embodiment of the present invention. The computer system 1000 includes any number of processors 1002 (also referred to as central processing units, or CPUs) that are coupled to storage devices including primary storage 1006 (typically a random access memory, or RAM), primary storage 1004 (typically a read only memory, or ROM). As is well known in the art, primary storage 1004 acts to transfer data and instructions uni-directionally to the CPU and primary storage 1006 is used typically to transfer data and instructions in a bi-directional manner. Both of these primary storage devices may include any suitable computer-readable media such as those described above. A mass storage device 1008 is also coupled bi-directionally to CPU 1002 and provides additional data storage capacity and may include any of the computer-readable media described above. Mass storage device 1008 may be used to store programs, data and the like and is typically a secondary storage medium such as a hard disk that is slower than primary storage. It will be appreciated that the information retained within the mass storage device 1008, may, in appropriate cases, be incorporated in standard fashion as part of primary storage 1006 as virtual memory. A specific mass storage device such as a CD-ROM 1014 may also pass data uni-directionally to the CPU.

CPU 1002 is also coupled to an interface 1010 that includes one or more input/output devices such as such as video monitors, track balls, mice, keyboards, microphones, touch-sensitive displays, transducer card readers, magnetic or paper tape readers, tablets, styluses, voice or handwriting recognizers, or other well-known input devices such as, of course, other computers. Finally, CPU 1002 optionally may be coupled to a computer or telecommunications network using a network connection as shown generally at 1012. With such a network connection, it is contemplated that the CPU might receive information from the network, or might output information to the network in the course of performing the method steps described herein. The above-described devices and materials will be familiar to those of skill in the computer hardware and software arts.

### 7. EXAMPLES

The following examples describe specific aspects of the present invention to illustrate the invention and also provide a description of the methods used to identify and use reference sets and investigation sets to aid those of skill in the art in understanding and practicing the invention. The examples should not be construed as limiting the present invention in any manner.

### Example 1

The MDDR (MDL Drug Data Report) which is a database of biologically active compounds with associated data, including activity classes was used as a reference for drug-like compounds (MDL Information Systems, Inc., 14600 Catalina St., San Leandro, Calif. 94577). Version 98.1 contains 92,604 entries. A subset of the MDDR was prepared using the following criteria, which are illustrated in FIG. 2.

First, only structures with a molecular weight of between about 200 Daltons to about 700 Daltons are included in the subset. A program called “StripSalt” was used to remove small-disconnected fragments such as salts from the SD files. (S. M. Muskal et al., U.S. application Ser. No. 09/114,694, filed on Jul. 13, 1998 which has been previously incorporated by reference).

Second only structures which consist entirely of C, N, O, H, S, P, F, Cl, Br and I atoms are included in the subset. Third, only structures that were sufficiently two dimensionally different from all other structures were included in the subset, thus eliminating close analogs that might bias the analysis. The measure of chemical identity chosen was the Tanimoto coefficient with the MDL 166 user keys, and compounds with a threshold value greater than about 0.8 were removed from the subset. The keys are 2D fragment-based descriptors, which are calculated automatically in MDL ISIS databases. (M. J. McGregor et al., J. Chem. Inf. Comput. Sci., 1997, 37, 443-448 which was previously incorporated herein by reference).

Finally, the compound activity class, as given in the activ_class and activ_index fields in the MDDR, indicates a unique target (enzyme or receptor). The file activity.txt, provided by MDL, which lists the classes was manually inspected to extract all such classes. Classes that had less than eight members, and compounds that belonged only to those classes, were eliminated from the subset. This procedure provided an MDDR subset of 9104 compounds (MDDR9104) and 152 classes that was used as the reference set for primary library design. Although compounds may belong to more than one class only 1083 compounds of the MDDR9104 belonged to multiple classes (11.9%).

Seven pharmacophore types (A, D, H, N, P, R and X) and six distance ranges (2.0-4.5 Å, 4.5-7.0 Å, 7.0-10.0 Å, 10.0-14.0 Å, 14.0-19.0 Å and 19.0-24.0 Å) were used to construct a basis set of 10, 549 pharmacophores, which were then used to fingerprint the MDDR9104. A single 3D molecular structure provided by the Corina program (J. Gasteiger et al., Tetrahedron Comp. Method., 1990, 3, 537; J. Sadowski et al., J. Chem. Inf. Comput. Sci. 1994, 34, 1000 which were previously incorporated by reference) was input into a proprietary program (M. J. McGregor et al., J. Chem. Inf. Comput. Sci., 1999, 39, 569 which was previously incorporated by reference) which assigns the pharmacophoric types to atoms, rotates about bonds to generate multiple conformations and builds the fingerprint by measuring distances between pharmacophoric groups. The output is a binary bitstring containing information about the pharmacophores presented by the molecule.

### Example 2

Molecules, which are similar according to a calculated property, should also be similar in biological activity. The following method was used as a measure of the discriminating power of a molecular descriptor, using the MDDR9104 data set classified into activity classes. Previous analyses that measure the discriminating power of a molecular descriptor have typically used only one target at a time (S. K. Kearsley et al., J. Chem. Inf. Comput. Sci., 1996, 36, 118 which was previously incorporated by reference).

First, all of the (n²−n)/2 pairwise intermolecular comparisons are made. Then the intermolecular comparisons are divided into comparisons made within classes and those made between classes. If a pair of compounds share at least one class when one compound belongs to several classes, both are in the same class. An assumption of the method is that compounds in the same class are more similar in biological activity than compounds in different classes. The pairwise intermolecular comparisons produce two distributions of molecular similarities. The difference in the means of the distributions of molecular similarity can be expressed in units of standard error by the formula:

t′=(X₁−X₂)/sqrt(s²₁/n₁+s²₂/n₂)

where for samples 1 and 2, X is the mean, s² is the variance and n is the sample size. The above expression follows the Student's t distribution for small samples while a normal distribution is followed for large samples. The statistic t′ is sometimes used as a test of significance for the difference between two distributions. The statistic is always highly significant in the results presented in Table 1. The absolute value of the statistic t′ is presented below. Generally, a larger absolute value implies superior discrimination. The statistic t′ can calculated for any data set that is assigned to classes and for any measure of similarity.

TABLE 1
t′ statistic using class assignments in the MDDR9104 set and
various molecular descriptors.

| | |
|---|---|
| Mol. Wt.: | t′ = 321.3 |
| MDL 166 keys Tanimoto: | t′ = 301.8 |
| Pharmacophore Fingerprint Tanimoto: | t′ = 455.8 |

| | |
|---|---|
| MSI₅₀/PCA | Pharmacophore Fingerprint/PCA |

| Dim | t′ | % var | t′ | % var |
|---|---|---|---|---|
| 1 | 330.1 | 63.5 | 306.0 | 22.9 |
| 1-2 | 344.5 | 72.8 | 403.2 | 30.2 |
| 1-3 | 359.7 | 79.1 | 445.1 | 35.4 |
| 1-4 | 351.1 | 84.8 | 455.2 | 39.2 |
| 1-5 | 372.1 | 88.9 | 442.1 | 42.6 |
| 1-6 | 365.9 | 92.0 | 434.9 | 45.2 |
| 1-7 | 369.9 | 94.0 | 434.6 | 47.0 |
| 1-8 | 371.7 | 95.8 | 440.3 | 48.6 |
| 1-9 | 374.0 | 96.8 | 440.9 | 49.9 |
| 1-10 | 374.9 | 97.6 | 441.9 | 51.0 |
| 1-11 | 374.9 | 98.1 | 442.7 | 52.0 |
| 1-12 | 375.7 | 98.5 | 446.3 | 53.0 |
| 1-13 | 375.3 | 98.9 | 447.2 | 53.8 |
| 1-14 | 374.8 | 99.2 | 446.8 | 54.5 |
| 1-15 | 374.7 | 99.4 | 447.9 | 55.2 |
| 1-16 | 374.6 | 99.5 | 448.4 | 55.8 |
| 1-17 | 374.6 | 99.6 | 448.7 | 56.4 |
| 1-18 | 374.6 | 99.7 | 447.8 | 56.9 |
| 1-19 | 374.6 | 99.7 | 448.1 | 57.5 |
| 1-20 | 374.7 | 99.8 | 447.3 | 57.9 |

Shown at the top of Table 1 is the t′ statistic for the MDDR9104 for three different molecular descriptors: molecular weight, a ID descriptor, the MDL 166 keys a 2D descriptor and pharmacophore fingerprints, a 3D descriptor. The Tanimoto coefficient was used to compare both the MDL 166 keys and the pharmacophore fingerprints while differences in molecular weight were used to compare the molecular weight descriptor.

Molecular weight was not expected to be a highly predictive descriptor. Surprisingly, molecular weight (t′=321.3) is superior to the MDL 166 keys (301.8). Both of these are outperformed by the pharmacophore fingerprint result (t′=455.8).

Results are also presented (lower section of Table 1) for a PCA analysis of the MSI₅₀ and pharmacophore fingerprint descriptors. The MSI₅₀ are 50 default descriptors in the software package Cerius2 from MSI (Molecular Simulations Inc., 9685 Scranton Road, San Diego, Calif. 92121-3752). The MSI descriptors vary in dimension. Some descriptors are calculated from a single 3D structure. However, none of the descriptors are calculated using multiple conformations. The MSI₅₀ is typical of descriptor sets used in many QSAR applications. The measure of similarity is Euclidean distance calculated in up to 20 dimensions.

The MSI₅₀ result reaches a maximum t′ of 375.7 at 12 dimensions (Table 1). However, at 5 principle components t′ is 372.1. The pharmacophore fingerprint result reaches a maximum t′ of 455.2 at 4 principle components (Table 1). The t′ values declines with the addition of more components.

Thus, the t′ results shown in FIG. 1 confirm the expected, but difficult to prove result, that 3D conformationally flexible descriptors provide superior discrimination over 3D one-conformer descriptors, which in turn outperform 2D descriptors. Significantly, the t′ results also show that the pharmacophore fingerprint /PCA result is comparable to the pharmacophore fingerprint/Tanimoto result. This result implies that the MDDR9104 can be meaningfully evaluated in a low dimensional space derived from transformation of pharmacophore fingerprints which simplifies calculational problems and aids in visualization in either 2 or 3 dimensions.

### Example 3

Principle Component Analysis was performed on the pharmacophore fingerprints of the MDDR9104 (see Example 1) to provide a low dimensional space suitable for pictorial representation. The pharmacophore fingerprints were treated as 10,549 independent variables and the 152 activity classes as dependent variables. The bits in the fingerprints were converted to the real numbers 0.0 (pharmacophore not present) and 1.0 (pharmacophore present) for the calculation. Activity for the MDDR9104 was entered as either 1.0, which signified binding to a particular activity class, or 0.0, which indicated the absence of binding to an activity class. The iterative NIPALS algorithm was used to transform the pharmacophore fingerprints to a low dimensional space suitable for visualization (P. Geladi, Anal. Chim. Acta, 1986, 185, which was previously incorporated by reference). The data were mean centered but not variance scaled. Table 1 (see Example 2) includes the variance for each component.

FIGS. 13 and 14 graphically illustrate the results of Principle Component Analysis of the MDDR9104. The plots depicted in these figures represent the coordinates of the T matrix shown in FIG. 11. Each compound in the MDDR9104 appears as a single point. The distribution of the MDDR9104 in components 1 and 2 is roughly wedge shaped with three significant prongs that roughly parallel the horizontal axis. FIGS. 13 and 14 show that the distribution of the MDDR9104 in two-dimensional chemical space is non-random with some regions much more densely populated than others.

Ideally, compounds with similar biological activities should be near one another in this chemical space. Conversely, compounds with different biological activities should be in different regions of chemical space. FIGS. 13A (components 1 and 2) and 13B (components 2 and 3) illustrate these principles by depicting the eight largest activity classes in the MDDR9104. FIGS. 13A and 13B provide a qualitative and visual representation of the separation of activity classes that was calculated by the t′ statistic in Example 2 above. Most activity classes are clustered in the same general region of chemical space, which supports the idea that the pharmacophore hypothesis has physical significance. Interestingly, most of the separation seems to be along the horizontal axis, which is the first principal component.

Determining the contribution of individual pharmacophores to the principal components is an important issue in Principle Component Analysis of the MDDR9104. FIG. 14A shows the plot of FIG. 13A color-coded according to the number of bits set in the pharmacophore fingerprint (i.e. the number of pharmacophores present in the molecule). A large number of bits set indicates a large, flexible and highly functionalized molecule. A strong separation in the first principal component is observed in FIG. 14A with the bit count increasing from right to left along the horizontal axis.

FIG. 14B shows the plot of FIG. 13A color coded according to the number of formal charges in the structure. A strong separation in the second principle component is observed. Compounds with negative charges and those with positive charges are located at the top and bottom of FIG. 14B respectively. Zwitterions and non-ionic compounds are clustered at the center of FIG. 14B.

Principle components 3 and 4 when colored appropriately and viewed on a 3D-computer graphics screen illustrate trends in hydrogen bonding, aromatic and hydrophobic groups of the MDDR9104. However these trends are more poorly defined than the bit count and charge examples illustrated in FIGS. 14A and 14B.

### Example 4

The MDDR9104 was chosen to be broadly representative of all bioactive molecules given currently available information. A test was devised to confirm whether the bioactive space produced by Principle Component Analysis of the MDDR9104 represents a universal bioactive space or if the bioactive space depends strongly on database content (See FIGS. 13 and 14 and Example 3).

Principle Component Analysis was performed on randomly selected subsets of the 152 classes of the MDDR9104. Growing subsets of compounds which belong to 19, 38, 57, 76, 95, 114 and 133 classes were created, where the larger sets are supersets of the smaller sets. This simulates the situation when active compounds for new targets are discovered and added to the MDDR database.

The Principle Component Analysis transformation is defined by the loadings matrix P (FIG. 11). A comparison of the P matrix was made for each subset with the preceding smaller subset and reported as a root mean square value (referred to as AP) for the first 4 principle components.

For example, Principle Component Analysis was performed on the compound set from 19 randomly selected classes. Another 19 randomly selected sets were added and Principle Component Analysis was repeated on the 38 randomly selected sets. The ΔP (19,38) value was calculated between the 19 randomly selected sets and the 38 randomly selected sets. Another 19 randomly selected classes were added to provide 57 randomly selected sets and the ΔP (38,57) calculated between the 38 randomly selected sets and the 57 randomly selected sets. The above process was repeated until it provided the complete MDDR9104 with 152 classes. The entire process was then repeated 10 times with different randomly selected sets. A low ΔP value as classes are added, especially in the later stages of the calculation, indicates that addition of new classes will not substantially change the nature of the bioactive space represented by the current MDDR9104.

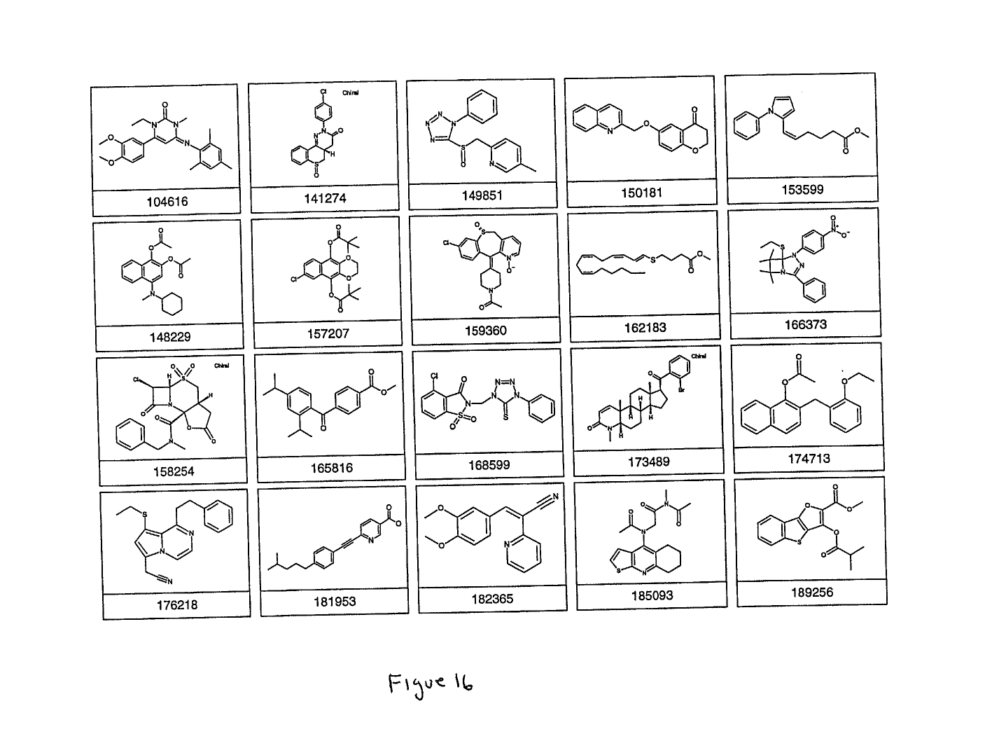

The results of the ΔP calculation are shown in FIG. 15. The value is a root mean square (RMS) of the summation of the first 4 principle components. Addition of later sets of classes provides a pronounced downward trend in the graph that approaches baseline, which indicates that addition of new classes in the future will not significantly change the nature of the bioactive space represented by the MDDR9104. This result indicates that the general features of ligand binding sites are representatively sampled by the MDDR9104 with the pharmacophore fingerprint descriptors. Note however, that a more detailed description of molecules (e.g., 4-point pharmacophores) may require more sampling.

### Example 5

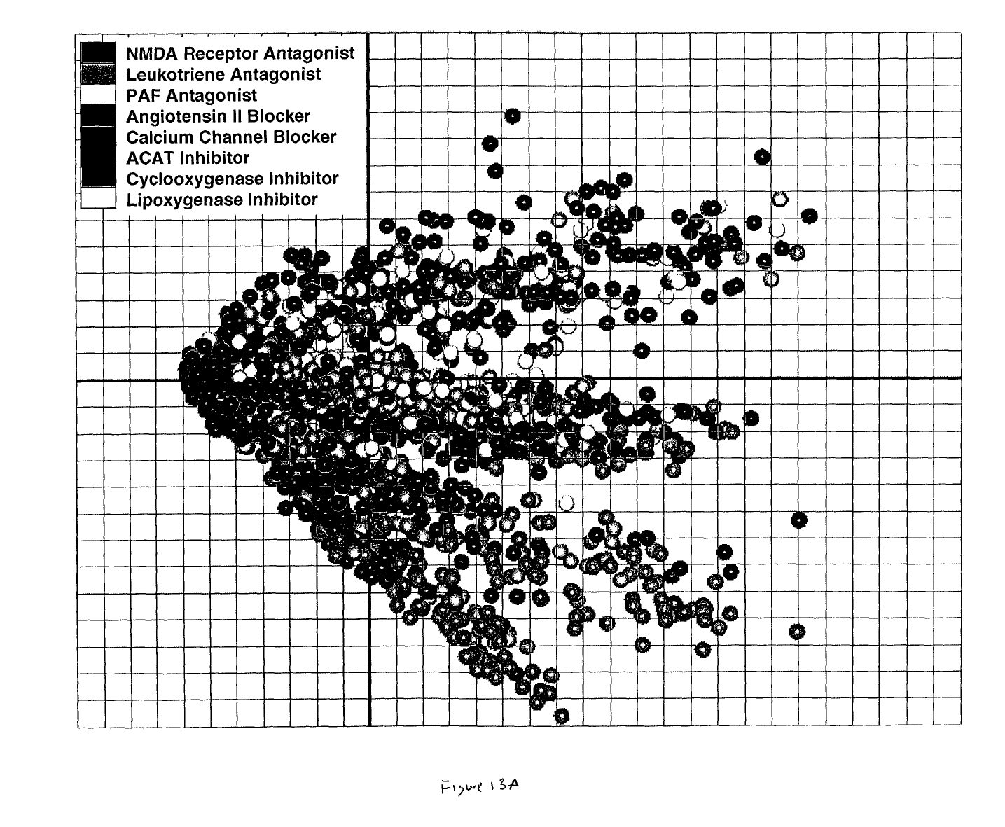

Eight scaffolds, illustrated in FIG. 12, that provide a diverse, commonly used set were used to construct libraries for combinatorial analysis. These scaffolds are well known to those of skill in the chemical arts. Each scaffold has 3 centers of diversity which may be enumerated with the same set of 20 surrogate building blocks to provide 8 libraries of 8000 molecules which simplifies library comparison. The building blocks are identical to the side chains of the 20 coded amino acids. The exception was proline, for which cyclopentyl glycine was substituted.

In other examples, the building blocks could be chosen for each scaffold based on synthetic feasibility and availability and could be of different chemical classes (e.g., amines, aldehydes etc.). In this example, the amino acid side chains were chosen because they are chemically diverse and biologically relevant.

A method was implemented to select subsets of building blocks to optimize a function such as an overlap function or molecular diversity function. The selection was done individually for each position in each scaffold. A set of 480 building blocks (i.e. 20 building blocks in 3 positions for 8 scaffolds) was selected. The selected building blocks were enumerated for each scaffold with a combinatorial constraint. Thus, all selected building blocks in the first position are enumerated with all selected building blocks in the second position etc. Initially, 50% of the building blocks were randomly selected which provided a subset of approximately 8000 selected molecules out of 64,000 possible molecules.

The algorithm commences with a random selection of building blocks and the function is calculated on the enumerated products. Then a randomly selected building block from the included set is excluded, and a randomly selected building block from the excluded set is included and the function is reevaluated. A Metropolis (probability) function is used to decide if the step is accepted or rejected, and the method proceeds iteratively until no further improvement is possible. The first function explored was overlap between the compound subset and the MDDR9104 in the bioactive space, which is referred to as the overlap function. Maximizing the overlap function optimizes the distribution of the enumerated compounds to most closely resemble the space represented by the MDDR9104.

The coordinate space resulting from the PCA calculation on the MDDR9104 set was divided into cubic cells of size 2.0 units in 3 dimensions. Principle Components 1, 2 and 3 were used in this analysis. Counts of the number of points (i.e. library compounds) with coordinates in each cell were made and scaled according to library size. Then a measure of the overlap of the distributions was made as follows:

Overlap=Σ{n1ₗ+n2ᵢ−abs(n1ᵢ−n2ₗ)}/(N1+N2)*100.0

Where:

N1=total number in set 1,

N2=total number in set 2,

n1ᵢ=number from set 1 in cell i,

n2ₗ=number from set 2 in cell i.

Essentially, this function is maximized when all cubic cells having members have same ratio of reference set members to investigation set members, and that ratio is equal to the ratio of total reference set members to total investigation set members.

The second function explored was the maxmin function which sums, for each molecule, the distance to its nearest neighbor (M. Snarey et al., J. Mol. Graphics Modeling, 1998, 15(6), 372 which was previously incorporated by reference). This produces a set when maximized, which spreads points as far apart as possible in the accessible space, and thus optimizes the molecular diversity of the library.

TABLE 2
Overlap of fully enumerated libraries with each other and with the MDDR9104 set.

| | MDDR | Lib1 | Lib2 | Lib3 | Lib4 | Lib5 | Lib6 | Lib7 | Lib8 |
|---|---|---|---|---|---|---|---|---|---|
| MDDR | 100 | 30 | 22 | 29 | 31 | 7 | 8 | 7 | 8 |
| Lib1 | | 100 | 39 | 44 | 34 | 9 | 12 | 10 | 14 |
| Lib2 | | | 100 | 32 | 18 | 18 | 18 | 22 | 23 |
| Lib3 | | | | 100 | 54 | 5 | 15 | 9 | 11 |
| Lib4 | | | | | 100 | 2 | 6 | 4 | 5 |
| Lib5 | | | | | | 100 | 14 | 37 | 52 |
| Lib6 | | | | | | | 100 | 13 | 19 |
| Lib7 | | | | | | | | 100 | 40 |
| Lib8 | | | | | | | | | 100 |

Table 2 shows the overlap of the fully enumerated libraries with one another and with the MDDR9104 in PCA space. The amount of overlap with the MDDR9104 represents the potential biological activity of the library. Considerable variation in overlap is observed as the percentage overlap of the first four libraries with the MDDR9104 varies between about 20% and about 30%. In contrast, the last four libraries have a percentage overlap with the MDDR9104 of less than 10% which indicates that these libraries are poor candidates for primary libraries. However, the last four libraries may be useful in more specialized applications such as intermediate or focused libraries. Importantly, the percentage overlap between libraries may be interpreted as a measure of similarity between different libraries. Once again a fair amount of variation exists (Table 2) and examination of the percentage overlap between libraries may be interpreted with reference to the scaffolds illustrated in FIG. 12.

Ten independent runs were performed in the building block selection simulation discussed above with different random number seeds for the overlap and maxmin functions. The results are presented as mean and standard deviation for the ten runs in Table 3. Optimization of the overlap function with the MDDDR9104 resulted in an initial (i.e. random) overlap of 29.7%(2.0)% and an optimized overlap of 52.6(0.3)%. As a point of reference, when the MDDR9104 set is split into two equal halves the percentage overlap between the two halves is only about 68.1% which indicates the difficulty of approaching 100%.

TABLE 3
Statistics for compound sets. Mean and standard deviation for: overlap function
with MDDR9104 (see text), number of compounds, molecular weight, clogP, number of
heavy atoms, number of bits (pharmacophores) in the fingerprint, number of rotatable
bonds, and the number of atoms per molecule assigned to the pharmacophore types.

| | libraries^{a} | | | | | | | | databases | | | |
|---|---|---|---|---|---|---|---|---|---|---|---|---|
| | 1.1.1.1.1. | | | | | | initial subset | | final subset | | | |
| | | | overlap | | maxmin | | MDDR9104 | | CMC | | ACD | |
| overlap | 29.7 | (2.0) | 52.6 | (0.3) | 26.4 | (0.7) | 100.0 | | 57.9 | | 48.0 | |
| compounds | 7990 | (286) | 7992 | (285) | 7974 | (287) | 9104 | | 6647 | | 213968 | |
| Mol. Wt. | 363 | (85) | 350 | (87) | 388 | (74) | 388 | (104) | 342 | (111) | 52 | (122) |
| clogP | −0.22 | (2.27) | 1.80 | (1.80) | 0.11 | (2.45) | 3.7 | (2.3) | 2.6 | (2.7) | 2.4 | (2.8) |
| atoms | 25.4 | (6.3) | 24.5 | (6.5) | 27.3 | (5.59) | 27.4 | (7.4) | 23.7 | (7.7) | 20.4 | (9.1) |
| bits | 899 | (622) | 806 | (633) | 1137 | (654) | 790 | (670) | 529 | (551) | 317 | (492) |
| rotbonds | 9.43 | (4.03) | 7.83 | (3.88) | 9.79 | (4.01) | 6.74 | (4.58) | 5.43 | (4.19) | 4.76 | (4.90) |
| X | 13.82 | (3.50) | 13.71 | (3.69) | 15.09 | (3.31) | 13.68 | (4.88) | 11.88 | (5.45) | 9.33 | (5.41) |
| A | 4.31 | (2.18) | 3.58 | (1.97) | 4.38 | (2.22) | 3.49 | (2.08) | 3.44 | (2.45) | 2.97 | (2.41) |
| D | 3.69 | (1.79) | 2.77 | (1.47) | 3.67 | (1.72) | 1.57 | (1.25) | 1.66 | (1.57) | 1.01 | (1.36) |
| H | 3.83 | (3.16) | 4.65 | (3.10) | 4.16 | (3.11) | 8.80 | (5.22) | 6.96 | (5.10) | 7.13 | (6.04) |
| N | 0.30 | (0.52) | 0.28 | (0.50) | 0.41 | (0.59) | 0.24 | (0.55) | 0.23 | (0.61) | 0.17 | (0.51) |
| P | 0.58 | (0.70) | 0.37 | (0.55) | 0.70 | (0.72) | 0.42 | (0.58) | 0.52 | (0.67) | 0.13 | (0.41) |
| R | 0.70 | (0.76) | 0.97 | (0.81) | 0.98 | (0.81) | 1.76 | (0.95) | 1.24 | (0.93) | 1.32 | (1.11) |

^{a}results calculated for 10 simulations

Table 3 gives some general statistics for initial and final combinatorial libraries and for the MDDR9104 and includes descriptors that were not part of the optimization calculation such as molecular weight, and clogP (Daylight Chemical Information Systems, Inc., 27401 Los Altos, Suite #370, Mission Viejo, Calif. 92691). In addition, two other reference sets, derived from MDL databases, are included for comparison: (i) CMC (filters: mol. wt. 150 to 750, atom type filter as for MDDR, salts removed), (i) ACD (filters: mol. wt. 1 to 1000, salts removed) (J. Greene, J. Chem. Inf. Comput. Sci., 1994, 34, 1297-1308 which is herein incorporated by reference).

The initial library subsets have a number of values such as the number of atoms and molecular weight similar to those found in the MDDR9104 set. The greatest discrepancies are an excessive number of H-bond donors, a relative lack of hydrophobic and aromatic groups and clogP values. In general, overlap optimization brings the statistics of the final libraries closer to the MDDR9104 statistics than optimization of the maxmin function. The overlap function also provides superior optimization of descriptors not explicitly part of the simulation (e.g. clogP) than the maxmin function in the final libraries.

TABLE 4
Frequency of occurrence of (i) scaffolds and (ii) building blocks in the
library subsets optimized for the overlap and the maxmin functions
(mean and s.d. for 10 simulations).
i) Scaffolds

Function

| Scaffold | overlap | | maxmin | |
|---|---|---|---|---|
| 1 | 1911 | (157) | 1455 | (113) |
| 2 | 1244 | (139) | 1694 | (111) |
| 3 | 1709 | (217) | 896 | (168) |
| 4 | 1444 | (158) | 463 | (65) |
| 5 | 463 | (91) | 1091 | (114) |
| 6 | 687 | (75) | 1389 | (133) |
| 7 | 219 | (56) | 302 | (70) |
| 8 | 313 | (69) | 684 | (108) |

ii) Building blocks

| | | Function | | | |
|---|---|---|---|---|---|
| Type | Description | overlap | | maxmin | |
| D | charged | 360 | (129) | 678 | (101) |
| E | charged | 258 | (132) | 662 | (96) |
| H | charged | 420 | (92) | 511 | (130) |
| K | charged | 124 | (90) | 539 | (123) |
| R | charged | 69 | (53) | 470 | (135) |
| Q | polar | 198 | (123) | 355 | (125) |
| N | polar | 191 | (104) | 188 | (147) |
| C | polar | 334 | (89) | 241 | (103) |
| S | polar | 149 | (116) | 144 | (115) |
| T | polar | 155 | (119) | 79 | (100) |
| A | small neutral | 514 | (121) | 247 | (142) |
| G | small neutral | 365 | (140) | 184 | (90) |
| Y | aromatic polar | 580 | (150) | 697 | (64) |
| W | aromatic polar | 486 | (116) | 756 | (66) |
| F | aromatic hydrophobic | 776 | (70) | 735 | (88) |
| L | aliphatic hydrophobic | 678 | (101) | 208 | (123) |
| M | aliphatic hydrophobic | 700 | (100) | 505 | (158) |
| (P) | aliphatic hydrophobic | 549 | (129) | 198 | (119) |
| I | aliphatic hydrophobic | 610 | (109) | 298 | (164) |
| V | aliphatic hydrophobic | 476 | (121) | 279 | (134) |

Table 4 shows the frequency counts for scaffolds and building blocks occurrence in the optimized libraries of Table 3. The relatively small standard deviations indicate that the results shown in Table 4 are reproducible. The first four scaffolds have a much greater frequency than the last four scaffolds in the libraries optimized for overlap with the MDDR9104. Significantly, this result confirms the overlap of the completely enumerated libraries shown in Table 2. The building block frequencies show a pronounced preference for hydrophobic and aromatic side chains and a trend against charged and polar side chains. The scaffold and building block frequency counts follow some of the same trends in the libraries optimized for the maxmin function, but tend to favor larger molecules in preference to the smaller ones.

One method for identifying holes in the space occupied by the optimized libraries was carried out by counting the number of MDDR9104 compounds in each cubic cell devoid of library compounds. A cell of the overlap-optimized subset with the highest number of MDDR9104 compounds had 44 such compounds, some of which are illustrated in FIG. 16. These MDDR9104 compounds are generally neutral molecules with aromatic rings and H-bond acceptors but no H-bond donors. Visual inspection of the scaffolds shown in FIG. 12 illustrates that all except one (the arnide scaffold #4) have at least one donor. Similarly examination of building block structure shows a lack of neutral side chains that have acceptors but not donors. Therefore, in retrospect, the inability of the optimized libraries to span certain portions of bioactive space represented by the MDDR9104 is easily appreciated but would have been difficult to predict a priori. The incorporation of new scaffolds and/or side chains in the analysis could presumably overcome this deficiency of the optimized combinatorial libraries.

The results above validate the utilization of MDDR9104/Principle Component Analysis space (i.e. bioactive space) for optimizing general properties of combinatorial libraries. Importantly, as shown above, comparison with MDDR9104/Principle Component Analysis space can also identify deficiencies in combinatorial libraries. Since combinatorial libraries comprised of the 20 amino acid side chains provide a skewed distribution in comparison to known bioactive compounds, the 20 amino acid side chains, when fully enumerated, may not be an optimum choice for ligand design.

While not wishing to be bound by theory two possible explanations may exist. First, protein binding sites tend to be hydrophobic, with hydrophilic residues reserved for the protein exterior. Second, ligands need to be complementary rather than congruent to the amino acids at the binding site. For example, if a proteins contain more H-bond donors, then a good ligand should contain more H bond acceptors.

Although the foregoing invention has been described in some detail to facilitate understanding, it will be apparent that certain changes and modifications may be practiced within the scope of the appended claims. For example, different basis sets could be used to fingerprint reference and investigation sets. Similarly, different reference and investigation sets could be used using the method of the current invention. Alternative methods such as genetic algorithms and neural networks can be applied to associate biological activity or any other activity exhibited by a collection of molecules to pharmacophore fingerprints. Different methods could be used to transform the pharmacophore fingerprints to a chemical space. Different criteria and procedures could be used to design a primary library from a reference set. Furthermore, it should be noted that there are alternative ways of implementing both the process and apparatus of the present invention. Accordingly, the present embodiments are to be considered as illustrative and not restrictive, and the invention is not to be limited to the details given herein, but may be modified within the scope and equivalents of the appended claims.

What is claimed is:

1. A method of identifying one or more regions of a defined activity in a chemical space, the method comprising:
receiving a reference set of compounds having members associated with the defined activity;
providing pharmacophore fingerprints of the members of the reference set, each fingerprint specifying a three dimensional superposition of pharmacophores from a basis set; and
associating the pharmacophore fingerprints of the members of the reference set with the defined activity so that at least one region of the chemical space associated with the defined activity is identified.

2. The method of claim 1, wherein the defined activity is a biological activity.

3. The method of claim 2, wherein the biological activity is a pharmacological activity.

4. The method of claim 1, wherein the defined activity is chosen from the group consisting of absorption, distribution, oral bioavailability, metabolism, and excretion.

5. The method of claim 1, wherein the reference set is comprised of pharmacologically active compounds.

6. The method of claim 1, wherein the reference set is or is derived from the compounds of the MDL Drug Data Report.

7. The method of claim 1, wherein the reference set is a subset of a database of pharmacologically active compounds.

8. The method of claim 7, wherein the subset is prepared by a method comprising:
selecting compounds from the database within a defined molecular weight range; and
selecting compounds from the database consisting of atoms selected from the group consisting of carbon, nitrogen, oxygen, hydrogen, sulfur, phosphorus, bromine, chlorine and iodine.

9. The method of claim 8, further comprising eliminating a compound from the subset when the Tanimoto coefficient between a structural representation of the compound and a structural representation of another compound in the database is greater than about a defined value.

10. The method of claim 1, wherein providing pharmacophore fingerprints for the members of the reference set comprises:
(a) receiving a three-dimensional representation of a compound of the reference set;
(b) assigning pharmacophoric types to positions in the three-dimensional representation of the compound, the pharmacophoric types specifying distinct chemical properties;
(c) choosing a current conformation of the compound;
(d) identifying matches between a current conformation of the compound and a basis set of pharmacophores, each pharmacophore in the basis set having at least three spatially separated pharmacophoric centers with associated pharmacophoric types; and
(e) creating the pharmacophore fingerprint from matches of the compound to members of the basis set.

11. The method of claim 10, wherein the pharmacophore types include at least a hydrogen bond acceptor, a hydrogen bond donor, a center with a negative charge, a center with a positive charge, a hydrophobic center, an aromatic center, and a default category that does not fall into any other specified pharmacophore type.

12. The method of claim 10, wherein identifying matches between a current conformation of the compound and a basis set of pharmacophores comprises identifying pharmacophores within the basis set that have pharmacophoric types located at the same relative positions as positions assigned the same pharmacophoric types in the current conformation of the compound.

13. The method of claim 10, wherein the pharmacophore fingerprint includes a bit sequence in which individual bits correspond to unique pharmacophores from the basis set.

14. The method of claim 1, wherein associating the pharmacophore fingerprints with the defined activity is performed with a regression technique.

15. The method of claim 1, wherein associating the pharmacophore fingerprints with the defined activity is performed by principal component analysis.

16. The method of claim 1, wherein associating the pharmacophore fingerprints with the defined activity is performed with a neural network or a genetic algorithm.

17. The method of claim 1, wherein associating the pharmacophore fingerprints with the defined activity transforms a representation of chemical space from a first representation including dimensions for members of the pharmacophore basis set to a second representation including dimensions for one or more principal components.

18. The method of claim 17, further comprising displaying the compounds of the reference set in the second representation of chemical structure space with the principal components as the dimension axes.

19. The method of claim 18, wherein the number of principal components used in displaying the compounds is two or three.

20. The method of claim 1, wherein associating the pharmacophore fingerprints reduces the dimensionality of the chemical space.

21. The method of claim 20 wherein associating the pharmacophore fingerprints provides a reduced set of orthogonal principal components.

22. The method of claim 21, wherein the principal components correspond to axes for a second representation of the chemical space.

23. A method for generating a library of compounds, the method comprising:
identifying one or more regions of a defined activity in a chemical space;
providing pharmacophore fingerprints of an investigation set of compounds for the library; and
identifying a subset of the investigation set of compounds having pharmacophore fingerprints falling within the one or more regions of the defined activity, the subset comprising the library.

24. The method of claim 23, wherein identifying the one or more regions of a defined activity in chemical space comprises:
receiving a reference set of compounds having members associated with the defined activity;
providing pharmacophore fingerprints of the members of the reference set, each fingerprint specifying a three dimensional superposition of pharmacophores from the basis set; and
associating the pharmacophore fingerprints of the members of the reference set with the defined activity so that at least one region of the chemical space associated with the defined activity is identified.

25. The method of claim 23, wherein identifying a subset of the investigation set of compounds comprises selecting a subset of the members of the investigation set that have substantial overlap with one or more regions of the defined activity in the chemical space.

26. The method of claim 25, wherein selecting the subset of the members of the investigation set comprises:
(a) randomly selecting a current subset of the members of the investigation set;
(b) calculating an overlap between the current subsets and the reference set within defined regions of the chemical space;
(c) selecting, based on calculated overlap, one of the current subset or a previous subset of the members of the investigation set;
(d) mutating a selected subset to change its membership; and
(e) repeating steps (b) through (d) until the overlap converges.

27. The method of claim 23, wherein the defined activity is a biological activity.

28. The method of claim 27, wherein the defined activity is a pharmacological activity.

29. The method of claim 28, wherein the library of compounds is a focused library and the activity is binding to a particular target.

30. The method of claim 28, wherein the library is a primary library and the one or more regions of a defined activity in chemical space include multiple therapeutic activities.

31. The method of claim 23, wherein the one or more regions of a defined activity in chemical space are the regions occupied by the MDL Drug Data Report.

32. The method of claim 24, wherein the reference set is or is derived from a database of pharmacologically active compounds.

33. The method of claim 32, wherein the subset is prepared by a method comprising:
selecting compounds from the database within a defined molecular weight range; and
selecting compounds from the database comprised of atoms selected from the group consisting of carbon, nitrogen, oxygen, hydrogen, sulfur, phosphorus, bromine, chlorine and iodine;

34. The method of claim 33, further comprising eliminating a compound from the subset when the Tanimoto coefficient between a structural representation of the compound and a structural representation of another compound in the database is greater than a defined value.

35. The method of claim 24, wherein providing pharmacophore fingerprints for the members of the investigation set comprises:
(a) receiving a three-dimensional representation of a compound of the investigation set;
(b) assigning pharmacophoric types to positions in the three-dimensional representation of the compound, the pharmacophoric types specifying distinct chemical properties;
(c) choosing a current conformation of the compound;
(d) identifying matches between a current conformation of the compound and a basis set of pharmacophores, each pharmacophore in the basis set having at least three spatially separated pharmacophoric centers with associated pharmacophoric types; and
(e) creating the pharmacophore fingerprint from matches of the compound to members of the basis set.

36. The method of claim 35, wherein the pharmacophore types include at least a hydrogen bond acceptor, a hydrogen bond donor, a center with a negative charge, a center with a positive charge, a hydrophobic center, an aromatic center, and a default category that does not fall into any other specified pharmacophore type.

37. The method of claim 24, wherein associating the pharmacophore fingerprint is performed with a regression technique.

38. The method of claim 24, wherein associating the pharmacophore fingerprint is performed by principal component analysis.

39. The method of claim 24, wherein associating the pharmacophore fingerprints with the defined activity transforms a representation of chemical space from a first representation including dimensions for members of the pharmacophore basis set to a second representation including dimensions for one or more principal components.

40. A computer program product comprising a machine readable medium on which is provided program code for identifying one or more regions of a defined activity in a chemical space, the program code specifying the following operations:
receiving a reference set of compounds having members associated with the defined activity;
providing pharmacophore fingerprints of the members of the reference set, each fingerprint specifying a three dimensional superposition of pharmacophores from the basis set; and
associating the pharmacophore fingerprints of the members of the reference set with at least the defined activity so that at least one region of the chemical structure space associated with the defined activity is identified.

41. The computer program product of claim 40, wherein the defined activity is a biological activity.

42. The computer program product of claim 40, wherein the reference set is or is derived from the compounds of the MDL Drug Data Report.

43. The computer program product of claim 40, wherein providing pharmacophore fingerprints for the members of the reference set comprises:
(a) receiving a three-dimensional representation of a compound of the reference set;
(b) assigning pharmacophoric types to positions in the three-dimensional representation of the compound, the pharmacophoric types specifying distinct chemical properties;
(c) choosing a current conformation of the compound;
(d) identifying matches between a current conformation of the compound and a basis set of pharmacophores, each pharmacophore in the basis set having at least three spatially separated pharmacophoric centers with associated pharmacophoric types; and
(e) creating the pharmacophore fingerprint from matches of the compound to members of the basis set.

44. The computer program product of claim 43, wherein the pharmacophore types include at least a hydrogen bond acceptor, a hydrogen bond donor, a center with a negative charge, a center with a positive charge, a hydrophobic center, an aromatic center, and a default category that does not fall into any other specified pharmacophore type.

45. The computer program product of claim 43, wherein identifying matches between a current conformation of the compound and a basis set of pharmacophores comprises identifying pharmacophores within the basis set that have pharmacophoric types located at the same relative positions as positions assigned the same pharmacophoric types in the current conformation of the compound.

46. The computer program product of claim 43, wherein the pharmacophore fingerprint includes a bit sequence in which individual bits correspond to unique pharmacophores from the basis set.

47. A computer program product comprising a machine readable medium on which is provided program code for generating a library of compounds, the program code specifying the following operations:
identifying one or more regions of a defined activity in a chemical space;
providing pharmacophore fingerprints of an investigation set of compounds for the library; and
identifying a subset of the investigation set of compounds having pharmacophore fingerprints falling within the one or more regions of the defined activity, the subset comprising the library.

48. The computer program product of claim 47, wherein identifying the one or more regions of a defined activity in chemical space comprises:
receiving a reference set of compounds having members associated with the defined activity;
providing pharmacophore fingerprints of the members of the reference set, each fingerprint specifying a three dimensional superposition of pharmacophores from a basis set; and
associating the pharmacophore fingerprints of the members of the reference set with the defined activity so that at least one region of the chemical space associated with the defined activity is identified.

49. The computer program product of claim 47, wherein identifying a subset of the investigation set of compounds comprises selecting a subset of the members of the investigation set that have a substantial overlap with the one or more regions of defined activity in the chemical space.

50. The computer program product of claim 47 further comprising transforming a representation of chemical space from a first representation including dimensions for members of the pharmacophore basis set to a second representation including dimensions for one or more principal components.

51. The computer program product of claim 47, wherein selecting the subset of the members of the investigation set comprises:
(a) randomly selecting a current subset of the members of the investigation set;
(b) calculating an overlap between the current subsets and the reference set within defined regions of the chemical space;
(c) selecting, based on calculated overlap, one of the current subset or a previous subset of the members of the investigation set;
(d) mutating a selected subset to change its membership; and
(e) repeating steps (b) through (d) until the overlap converges.

52. A computer program product comprising a machine readable medium on which is provided a representation of a chemical space,
which representation includes one or more principal components derived from pharmacophore fingerprints and associated activities for a plurality of compounds from a reference set of compounds, and
which representation of the chemical space identifies one or more regions of a defined activity.

53. The computer program product of claim 52, wherein the defined activity is a biological activity.

54. The method of claim 25, wherein selecting the subset of the members of the investigation set comprises:
(a) randomly selecting subsets of the members of the investigation set;
(b) calculating an overlap between the subsets and the reference set within defined regions of the chemical space;
(c) randomly selecting a current subset;
(d) mutating the current subset to change membership;
(e) calculating an overlap between the current subset and the reference set within defined regions of the chemical space;
(f) determining whether the mutation of the current subset is accepted; (g) repeating steps (c) through (e) until mutation of the current subset is rejected;
(h) evaluating whether the overlap between the current subset and the reference set has converged;
(i) repeating steps (c ) through (g) until overlap between the current subset and the reference set converges;
(j) repeating steps (c) through (i) with until all subsets of the members of the investigation set that have substantial overlap with one or more regions of the defined activity in the chemical space have been identified.

55. The computer program product of claim 47, wherein selecting the subset of the members of the investigation set comprises:
(a) randomly selecting subsets of the members of the investigation set;
(b) calculating an overlap between the subsets and the reference set within defined regions of the chemical space;
(c) randomly selecting a current subset;
(d) mutating the current subset to change membership;
(e) calculating an overlap between the current subset and the reference set within defined regions of the chemical space;
(f) determining whether the mutation of the current subset is accepted;
(g) repeating steps (c) through (e) until mutation of the current subset is rejected;
(h) evaluating whether the overlap between the current subset and the reference set has converged;
(i) repeating steps (c ) through (g) until overlap between the current subset and the reference set converges;
(j) repeating steps (c) through (i) with until all subsets of the members of the investigation set that have substantial overlap with one or more regions of the defined activity in the chemical space have been identified.
